(19) United States
(12) Patent Application Publication
(10) Pub. No.: US 2025/0333713 A1
(43) Pub. Date: Oct. 30, 2025

(54) Mechanical Lysis

(71) Applicant: ASCEND ADVANCED THERAPIES LIMITED, Altrincham, Cheshire (GB)

(72) Inventors: Ahmed Samir Ibrahim Aly Youssef, Planegg (DE); Markus Hoerer, Planegg (DE)

(73) Assignee: ASCEND ADVANCED THERAPIES LIMITED, Altrincham, Cheshire (GB)

(21) Appl. No.: 18/855,548

(22) PCT Filed: Apr. 13, 2023
(86) PCT No.: PCT/GB2023/050996

§ 371 Date: Oct. 9, 2024

(30) Foreign Application Priority Data
Apr. 13, 2022 (GB) 2205514.9

(51) Int. Cl. C12N 7/00; C12N 15/86

(52) U.S. Cl. CPC C12N 7/00; C12N 15/86; C12N 2750/14143; C12N 2750/14151

(57) ABSTRACT

The present invention relates to methods for producing a preparation comprising recombinant AAV (rAAV), such methods comprising a step of mechanical lysis on mammalian producer cells, and methods for increasing the viral genome titre and/or capsid titre of a preparation comprising recombinant adeno-associated virus, related uses, and preparations obtained by or obtainable by the methods.

### CROSS REFERENCE TO RELATED APPLICATIONS

The present application is the US National Phase Entry of International Patent Application Serial No. PCT/GB2023/050996, filed on Apr. 13, 2023, which claims priority to United Kingdom Patent Application Serial No. GB2205514.9 filed Apr. 13, 2022, all of which are incorporated herein by reference in their entirety.

### SEQUENCE LISTING

The present application is being filed along with a Sequence Listing in electronic format that is an XML file. The Sequence Listing is provided herein as a file entitled ASCEND-P001_SEQLIST.xml, created on Jun. 30, 2025, which is 80,169 bytes in size and is hereby incorporated by reference in its entirety.

### FIELD OF THE INVENTION

The present invention relates to methods for producing a preparation comprising recombinant AAV (rAAV) and methods for increasing the viral genome titre and/or capsid titre of a preparation comprising recombinant adeno-associated virus, related uses, and preparations obtained by or obtainable by the methods.

### BACKGROUND TO THE INVENTION

Recombinant adeno-associated virus (rAAV) vectors have considerable potential for gene therapy due to their promising safety profile and their ability to transduce many tissues in vivo. However, it remains difficult to produce high quality rAAV at high yield. Methods of rAAV production that lead to higher capsid/viral genome titres are advantageous, as they allow for greater amounts of therapeutic AAV to be produced with greater efficiency. Similarly, improving the proportion of produced rAAV particles containing (“full”) rather than lacking (“empty”) a complete recombinant vector genome, as measured by the viral genome/capsid ratio, is advantageous as, at least in the case of AAV used for gene therapy, increasing viral genome/capsid ratio ensures that an increased amount of therapeutic transgene is present per dose of AAV preparation.

However, production of AAV is still quite difficult and scale-up of production to an industrial scale has been accomplished only to a limited degree. AAV are generally produced by transforming cells (such as producer cells) with genetic material encoding the AAV, culturing the cells under conditions suitable for the AAV to propagate within the cells and then harvesting the AAV from the cells. However, there is currently a lack of scalable methods for obtaining AAV from cells which maintain high viral genome and capsid yields. For example, while freeze/thaw methods of producing preparations comprising recombinant AAV result in high viral genome yields, capsid yields and viral genome/capsid ratio at laboratory scale, they are not practical to use at the industrial scale and they are time-consuming. Other methods relying on lysing the cell membrane of the producer cells by detergents result in poor yield and furthermore require that the detergents are completely removed during the down-stream process. Methods of harvesting AAV using alternative mechanical lysis methods generally result in low viral genome and capsid yields.

### SUMMARY OF THE INVENTION

It is an object of the present disclosure to provide methods and materials for generating high titre preparations of recombinant adeno-associated virus (rAAV). Various methods for the generation and processing of rAAV particles in mammalian cells are described in detail below, and illustrations of the use of such techniques are provided in the Examples following.

In particular, the present invention provides a method for producing a preparation comprising recombinant AAV, wherein the method comprises a step of performing mechanical lysis on mammalian producer cells comprising the recombinant AAV at a pressure of 7 kilo pounds per square inch (kpsi) or greater. The use of mechanical lysis methods like microfluidisation allow for effective industrial scale harvesting of rAAV. Furthermore, the present inventors surprisingly found that methods using mechanical lysis at pressures higher than those recommended for mechanical lysis of human cells can be used to obtain rAAV preparations with higher viral genome and/or capsid titre. Further, the present inventors found that combination of a step of mechanical lysis with a step of depth filtration increased viral genome/capsid ratio while also decreasing the turbidity of the resulting preparation. Methods of the present application will therefore be useful in the industrial production of rAAV preparations for use in, e.g., gene therapy.

The present application demonstrates that a method comprising a step of performing mechanical lysis on mammalian producer cells comprising recombinant AAV at a pressure of 7 kpsi or greater results in preparations comprising recombinant AAV with increased viral genome and capsid yields. The present application also demonstrates that methods additionally comprising a step of depth filtration result in preparations comprising recombinant AAV with reduced turbidity and increased viral genome/capsid ratios.

Accordingly, in a first aspect of the invention, there is provided a method for producing a preparation comprising recombinant adeno-associated virus (AAV), wherein the method comprises a step of performing mechanical lysis on mammalian producer cells comprising the recombinant AAV at a pressure of 7 kilo pounds per square inch (kpsi) or greater.

In a second aspect of the invention, there is provided a method for increasing the viral genome titre and/or capsid titre of a preparation comprising recombinant adeno-associated virus (AAV), wherein the method comprises a step of performing mechanical lysis on mammalian producer cells comprising the recombinant AAV at a pressure of 7 kilo pounds per square inch (kpsi) or greater.

In a third aspect of the invention, there is provided a use of mechanical lysis for producing a preparation comprising recombinant adeno-associated virus (AAV), wherein the mechanical lysis is performed on mammalian producer cells at a pressure of 7 kilo pounds per square inch (kpsi) or greater.

In a fourth aspect of the invention, there is provided a use of mechanical lysis for increasing the viral genome titre and/or capsid titre of a preparation comprising recombinant adeno-associated virus (AAV), wherein the mechanical lysis is performed on mammalian producer cells comprising the recombinant AAV at a pressure of 7 kilo pounds per square inch (kpsi) or greater.

In a fifth aspect of the invention, there is provided a method or use of the invention further comprising a step of depth filtration.

In a sixth aspect of the invention, there is provided a preparation comprising recombinant AAV obtainable by the methods or uses of the invention.

In a seventh aspect of the invention, there is provided a preparation comprising recombinant AAV obtained by the methods or uses of the invention.

### DESCRIPTION OF THE FIGURES

FIG. 1A: Normalised capsid titres following cell lysis. Shown are the rAAV capsid titres obtained from mechanical lysis of HEK293 suspension cells at three different pressure settings (5 kpsi, 10 kpsi and 20 kpsi). Results are normalised to 5 kpsi at 100%. Capsid titres were determined by ELISA.

FIG. 1B: Normalised viral genome titres following cell lysis. Shown are the rAAV viral genome titres obtained from mechanical lysis of HEK293 suspension cells at three different pressure settings (5 kpsi, 10 kpsi and 20 kpsi). Results are normalised to 5 kpsi at 100%. Viral genome titres were determined by qPCR.

FIG. 1C: Normalised viral genome/capsid titres following cell lysis. Shown is the ratio of the viral genome (vg) per capsid (cap) titres obtained from mechanical lysis of HEK293 suspension cells at three different pressure settings (5 kpsi, 10 kpsi and 20 kpsi). Results are normalised to 5 kpsi at 100%. Viral genome titres were determined by qPCR and capsid titres by ELISA.

FIG. 2A: Normalised capsid titres following cell lysis. Shown are the rAAV capsid titres obtained from mechanical lysis of HEK293 suspension cells at six different pressure settings as indicated. Results are normalised to 5 kpsi at 100%. Capsid titres were determined by ELISA.

FIG. 2B: Normalised viral genome titres following cell lysis. Shown are the rAAV viral genome titres obtained from mechanical lysis of HEK293 suspension cells at six different pressure settings ranging from 2 kpsi to 40 kpsi. Results are normalised to 5 kpsi at 100%. Viral genome titres were determined by qPCR.

FIG. 2C: Normalised viral genome per capsid titres following cell lysis. Shown is the ratio of the viral genome (vg) per capsid (cap) titres obtained from mechanical lysis of HEK293 suspension cells at five different pressure settings between 5 kpsi and 40 kpsi. Results are normalised to 5 kpsi at 100%. Viral genome titres were determined by qPCR and capsid titres by ELISA.

FIG. 3: Depth Filtration—C0SP and C0HC filters. After microfluidisation at 20 kpsi, depth filtration was tested to decrease the turbidity, using a synthetic (C0SP) or an organic (C0HC) filter with micron rating (0.2-1.1 μm). (A) Capsid titres obtained following depth filtration using a synthetic (C0SP) or an organic (C0HC) filter. Capsid titres were determined using AAV2-ELISA. Results shown are normalised against the control without depth filtration (microfluidisation only). (B) Viral genome titres obtained following depth filtration using a synthetic (C0SP) or an organic (C0HC) filter. Viral genome titres were determined using qPCR. Results shown are normalised against the control without depth filtration (microfluidisation only). (C) Normalised viral genome per capsid titres following cell lysis and depth filtration. Shown is the ratio of the viral genome (vg) per capsid (cap) titres. Results are normalised to the control at 100%. Viral genome titres were determined by qPCR and capsid titres by ELISA.

### DETAILED DESCRIPTION

### General Definitions

Unless defined otherwise, technical and scientific terms used herein have the same meaning as commonly understood by a person skilled in the art to which this invention belongs.

In general, the term “comprising” is intended to mean including but not limited to. For example, the phrase “a method comprising a step of mechanical lysis” should be interpreted to mean that the method includes a step of mechanical lysis, but the method may comprise further steps.

The terms “protein” and “polypeptide” are used interchangeably herein, and are intended to refer to a polymeric chain of amino acids of any length.

The terms “nucleic acid molecule” “nucleic acid sequence”, “polynucleotide” and “nucleotide sequence” are used interchangeably herein, and are intended to refer to a polymeric chain of nucleotides of any length e.g. deoxyribonucleotides, ribonucleotides, or analogs thereof. For example, the polynucleotide may comprise DNA (deoxyribonucleotides) or RNA (ribonucleotides). The polynucleotide may consist of DNA. The polynucleotide may be mRNA. Since the polynucleotide may comprise RNA or DNA, all references to T (thymine) nucleotides may be replaced with U (uracil).

For the purpose of this invention, in order to determine the percent identity of two sequences (such as two polynucleotide or two polypeptide sequences), the sequences are aligned for optimal comparison purposes (e.g., gaps can be introduced in a first sequence for optimal alignment with a second sequence). The nucleotides or amino acids at each position are then compared. When a position in the first sequence is occupied by the same amino acid or nucleotide as the corresponding position in the second sequence, then the amino acids or nucleotides are identical at that position. The percent identity between the two sequences is a function of the number of identical positions shared by the sequences (i.e., % identity=number of identical positions/total number of positions in the reference sequence×100).

Typically the sequence comparison is carried out over the length of the reference sequence. For example, if the user wished to determine whether a given (“test”) sequence is 95% identical to SEQ ID NO: 1, SEQ ID NO: 1 would be the reference sequence. To assess whether a sequence is at least 80% identical to SEQ ID NO: 1 (an example of a reference sequence), the skilled person would carry out an alignment over the length of SEQ ID NO: 1, and identify how many positions in the test sequence were identical to those of SEQ ID NO: 1. If at least 80% of the positions are identical, the test sequence is at least 80% identical to SEQ ID NO: 1. If the sequence is shorter than SEQ ID NO: 1, the gaps or missing positions should be considered to be non-identical positions.

The skilled person is aware of different computer programs that are available to determine the homology or identity between two sequences. For instance, a comparison of sequences and determination of percent identity between two sequences can be accomplished using a mathematical algorithm. In an embodiment, the percent identity between two amino acid or nucleic acid sequences is determined using the EMBOSS Needle Pairwise Sequence Alignment tool.

Herein, the term “plasmid” is intended to refer to a nucleic acid molecule that can replicate independently of a cell chromosome. The term “plasmid” is intended to cover circular nucleic acid molecules and linear nucleic acid molecules. Furthermore, the term “plasmid” is intended to cover bacterial plasmids, but also cosmids, minicircles (Nehlsen,
K., Broll S., Bode, J. (2006), Gene Ther. Mol. Biol., 10: 233-244; Kay, M. A., He, C.-Y,
Chen, Z.-H. (2010), Nature Biotechnology, 28: 1287-1289) and ministrings (Nafissi N,

Alqawlaq S, Lee E A, Foldvari M, Spagnuolo P A, Slavcev R A. (2014), Mol Ther Nucleic 15 Acids, 3:e165). Optionally, the plasmid is a circular nucleic acid molecule. Optionally, the plasmid is a nucleic acid molecule that is of bacterial origin.

The term “corresponding method” refers to a method that is identical to a different method, but for one feature. For example, a “corresponding method comprising a step of performing mechanical lysis at a pressure of 5 kpsi” is a method which is identical to a method of the invention, except that mechanical lysis occurs at a pressure of 5 kpsi.

The term “around” in relation to a reference numerical value and its grammatical equivalents as used herein can include the numerical value itself and a range of values plus or minus 10% from that numerical value. For example, the term “around” in relation to a reference numerical value can also include a range of values plus or minus 10%, 9%, 8%, 7%, 6%, 5%, 4%, 3%, 2%, or 1% from that value. For example, reference to a pressure of “around” 20 kpsi may refer to a pressure of 18-22 kpsi.

The term “between” in relation to a pair of reference numerical values and its grammatical equivalents as used herein can include the numerical values themselves and the range of values between the reference numerical values. For example, the term “between 10 kpsi and 40 kpsi” may refer to a pressure of 10 kpsi, 40 kpsi, or any value falling within the range 10-40 kpsi.

The singular forms “a”, “an”, and “the” include plural referents unless the content clearly dictates otherwise. Thus, for example, reference to “an AAV cap gene” includes two or more instances or versions of such cap genes.

All publications, patents and patent applications cited herein, whether supra or infra, are hereby incorporated by reference in their entirety.

### AAV Production Assay

An AAV production assay may be used to test whether certain features of the methods or uses of the invention allow for suitable AAV production.

The user provides a “reference” mammalian producer cell that comprises sufficient genetic material to produce recombinant AAV when cultured under conditions suitable for the production of rAAV.

For example, the user provides a reference mammalian producer cell comprising wild type Adenovirus 5 helper genes encoding E2A, E4 and VA RNA I and II, i.e. the adenovirus helper genes comprised within the Adenovirus 5 genome with Genbank Sequence ID: AC_000008.1 (SEQ ID NO: 2). Details of the nucleic acid positions in SEQ ID NO: 2 which encode these genes are set out in more detail below under the heading “helper genes”. The reference mammalian producer cell also comprises a wild type rep gene encoding Rep 40, Rep 52, Rep 68 and Rep 78 and the rep promoters p5, p19 and p40, i.e. the sequences comprised within nucleotides 200-2252 of AAV2 genome with Genbank Sequence ID: NC_001401.2 (SEQ ID NO: 1).

The reference mammalian producer cell also comprises a wild type cap gene operably linked to a wild type cap gene promoter comprising p5, p19 and p40, i.e. the cap gene comprised within SEQ ID NO: 1 (nucleotides 5961-8171 of SEQ ID NO: 1). The vector plasmid further comprises a transgene flanked by two AAV2 ITRs, i.e. the ITRs comprised within nucleotides 1-145 and 4535-4679 of SEQ ID NO: 1.

The user then provides a “test” mammalian producer cell that is based on the reference mammalian producer cell, but has a single change relating to a characteristic that the user wishes to test. For example, if the user wishes to see whether a given Rep protein was functional, the user could swap out the rep gene of the “reference mammalian producer cell” and replace it with the test rep protein to provide a “test mammalian producer cell”.

The user then compares the ability of the reference mammalian producer cell and the test mammalian producer cell to allow for production of rAAV. To do this, the user can incubate the reference mammalian producer cells and test producer cells for a period of time suitable for the rAAV production to occur. The yield of rAAV produced from the reference mammalian producer cell and the test mammalian producer cell may then be harvested and measured using qPCR to quantify the number of vector genomes. For example, qPCR may be used to determine the number of instances of nucleic acid molecules comprising a component of the vector genome, such as a promoter sequence, that are produced in the test mammalian producer cells compared to the reference mammalian producer cells. Alternatively, the comparative yield of particles may be determined, for example by an anti-capsid ELISA.

### Mammalian Producer Cells

The present invention relates to methods and uses comprising a step of performing mechanical lysis on mammalian producer cells comprising recombinant AAV at a pressure of 7 kpsi or greater.

AAV are viruses that are useful in applications such as gene therapy, and replicate in human cells. It is typical to employ a host or “producer” cell for rAAV vector replication and packaging. Such a producer cell (suitably a mammalian host cell) generally comprises or is modified to comprise several different types of components for rAAV production. Thus, mammalian cells may be used to produce AAV in quantities suitable for harvesting the AAV (in a preparation comprising recombinant AAV). Cells that are suitable for propagation of AAV may be referred to as “mammalian producer cells”. The mammalian producer cells used in the methods and uses of the invention comprise recombinant AAV. For example, the mammalian producer cells may comprise AAV because they comprise sufficient genetic material for AAV to propagate and/or because they have been cultured under conditions suitable for the production of rAAV. The skilled person can easily determine whether a given cell is suitable for the production of AAV using the assay described under the heading AAV production assay by using the given cell as a “test” mammalian producer cell.

In one embodiment of the present invention, the mammalian producer cells are human cells. Optionally, the mammalian producer cells are human kidney cells. Mammalian producer cells are human cells or human kidney cells if they are derived from human cells or human kidney cells, for example the HEK293 immortalised human kidney cells should be considered both human cells and kidney cells.

Optionally, the mammalian producer cells are HEK293 cells, HEK293T cells, HEK293SF cells, HEK293-F cells, HEK293-derived cells, CHO cells, HeLa cells, HeLa-derived cells, HeLa S3 cells, HEK293EBNA cells, CAP cells, CAP-T cells, AGE1.CR cells, PerC6 cells, C139 cells, EB66 cells, BHK cells, COS cells, Vero cells, A549 cells, or other cells derived from any of these cells. In one embodiment of the present invention, the mammalian producer cells are selected from the group consisting of HEK293 cells, HEK293T cells, HEK293-F cells, HEK293SF cells, HEK293-derived cells, CHO cells, HeLa cells, HeLa S3 cells, HEK293EBNA cells, CAP cells, CAP-T cells, AGE1.CR cells, PerC6 cells, C139 cells, EB66 cells, BHK cells, COS cells, Vero cells, and A549 cells. In one embodiment of the present invention, the mammalian producer cells are HEK293 cells, HEK293T cells, HEK293SF cells, HEK293-F cells, HEK293-derived cells, CHO cells, HeLa cells, HeLa S3 cells, HEK293EBNA cells, CAP cells, CAP-T cells, AGE1.CR cells, PerC6 cells, C139 cells, EB66 cells, BHK cells, COS cells, Vero cells, or A549 cells. Optionally, the mammalian producer cells are HEK293 or HEK293T cells.

Optionally, the mammalian producer cells are of a cell type that is suited to suspension or adherent cell culture or were cultured in suspension or adherent cell culture before the step of performing mechanical lysis. Optionally, the mammalian producer cells are of a cell type that is suited to suspension cell culture. Optionally, the mammalian producer cells were cultured in suspension culture before the step of performing mechanical lysis. As set out in more detail below under the heading “Cell culture systems” cells that are cultured in suspension culture tend to have a different morphology compared to cells that are cultured in adherent culture. In particular, cells cultured in adherent culture tend to be flatter and less rounded that cells that are cultured in suspension culture.

### Mechanical Lysis

Once mammalian producer cells comprising recombinant AAV have been obtained, it is necessary to harvest the recombinant AAV from the cells, i.e. separate the preparation recombinant AAV from the mammalian producer cell material. That can be achieved by performing a step of mechanical lysis, which can be used to lyse the mammalian producer cells and allow the recombinant AAV to be released from the mammalian producer cells.

The term “mechanical lysis” refers to methods of lysing cell membranes, wherein the cell membrane is physically broken apart using shear force. Methods of mechanical lysis include but are not limited to homogenization, sonication, and microfluidisation. Optionally, the mechanical lysis is not carried out by freeze/thaw. Optionally, the mechanical lysis is carried out by microfluidisation.

Microfluidisation refers to the use of fluid pressure to create large shear forces. A microfluidisation machine is typically used to perform microfluidisation. Microfluidisation machines may comprise a specifically designed chamber through which a fluid sample passes. The microfluidisation machine may be a Constant System LTD—CF1 (cylinder diameter 18 mm). Other microfluidisation devices suitable for cell lysis of mammalian cells are known to the person skilled in the art. Typically, a fluid sample is pumped through the chamber at a specified pressure. This pressure can be set by the user when operating a microfluidisation machine. Passing through the chamber at pressure results in large shear forces throughout the volume of fluid in the chamber, which are capable of, e.g. lysing mammalian cells. Optionally, the geometry of the chamber is designed to generate high shear forces by restricting the fluid to a sub-millimetre scale, i.e. by forcing the fluid through sub-millimetre passages. For example, cells (such as the mammalian producer cells used in the methods and uses of the invention) may enter the system via an inlet reservoir and be pulled into a constant pressure pumping system which pushes the cells through a small fixed orifice at high velocity, causing cell disruption. The sample may then hit a cooling head and spread radially, then vertically down a cooled heat exchange surface of the cooler head. The disrupted cells may then leave the device through an outlet.

A “pass” may refer to the mammalian producer cells being pumped through a high pressure chamber in a microfluidisation machine once. At lower pressures, it may be necessary to perform more than one pass through the microfluidisation chamber in order to achieve suitable levels of lysis of the mammalian producer cells. At higher pressures, it may only be necessary for the step of microfluidisation to comprise a single pass.

In one embodiment, the step of mechanical lysis is carried out by a step of microfluidisation consisting of three or fewer passes, e.g. the mammalian producer cells are passed through a high pressure chamber in a microfluidisation machine no more than three times. Whilst the methods and uses of the invention may comprise more than one mechanical lysis step, in methods or uses in which the step of microfluidisation consists of three passes or fewer, the method or use may not involve more than three passes of microfluidisation. In another embodiment, the step of mechanical lysis is carried out by a step of microfluidisation consisting of two or fewer passes. In another embodiment, the step of mechanical lysis is carried out by a step of microfluidisation consisting of one pass.

### Preparation Comprising Recombinant AAV

A “preparation” is a solution produced by any of the methods or uses of the present invention. Optionally, a preparation may comprise recombinant AAV. “Recombinant AAV” or “rAAV” refers to AAV particles, i.e. particles comprising an AAV genome (such as a vector genome) and an AAV capsid. The rAAV may be of any serotype. Optionally, the rAAV may comprise a genome of one serotype and a capsid of another serotype. The AAV capsid may comprise proteins from more than one serotype, otherwise known as a pseudotyped capsid. The preparation may comprise further components such as pharmaceutically acceptable excipients as discussed in more details below.

### At a Pressure of . . .

Methods of mechanical lysis may be carried out at a specific pressure or range of pressures. “Pressure” in the context of the present invention refers to the pressure at which a step of mechanical lysis is performed. For example, when using microfluidisation, the step of mechanical lysis occurs at a “pressure of 7 kpsi or greater” if the pressure at which the microfluidisation machine is set by the user is 7 kpsi or greater. The pressure of the microfluidisation machine as set by the user is the pressure at which the sample passes through the high-pressure chamber. The greater the pressure, the greater the shear forces which the sample experiences in the high-pressure chamber. High shear forces may result in lysis of mammalian producer cells, thus releasing recombinant AAV. In one aspect of the present invention, the step of performing mechanical lysis on mammalian producer cells comprising recombinant AAV occurs at a pressure of 7 kpsi or greater. Suitably, the step of performing mechanical lysis on mammalian producer cells comprising recombinant AAV occurs at a pressure of around 7 kpsi or greater, which is generated using a Constant System LTD—CF1 (cylinder diameter 18 mm) microfluidisation machine. Suitably, mechanical lysis on mammalian producer cells comprising recombinant AAV occurs at shear rates and/or shear stress generated by a Constant System LTD—CF1 (cylinder diameter 18 mm) microfluidisation machine at pressure of around 7 kpsi or greater. Suitably, in the methods or uses of the invention other mechanical lysis systems, such as other microfluidisation devices, can be used which generate an equivalent shear rate and/or shear stress to that generated by a Constant System LTD—CF1 (cylinder diameter 18 mm) microfluidisation machine at pressure of around 7 kpsi or greater.

In another embodiment, the step of performing mechanical lysis on mammalian producer cells comprising recombinant AAV occurs at a pressure of 10 kpsi or greater, 11 kpsi or greater, 12 kpsi or greater, 13 kpsi or greater, 14 kpsi or greater, 15 kpsi or greater, 16 kpsi or greater, 17 kpsi or greater, 18 kpsi or greater, 19 kpsi or greater, 20 kpsi or greater, 21 kpsi or greater, 22 kpsi or greater, 23 kpsi or greater, 24 kpsi or greater, 25 kpsi or greater, 26 kpsi or greater, 27 kpsi or greater, 28 kpsi or greater, 29 kpsi or greater, 30 kpsi or greater, 31 kpsi or greater, 32 kpsi or greater, 33 kpsi or greater, 34 kpsi or greater, or 35 kpsi or greater.

In another embodiment, the step of performing mechanical lysis on mammalian producer cells comprising recombinant AAV occurs at a pressure of around 7 kpsi or greater, around 8 kpsi or greater, around 9 kpsi or greater, around 10 kpsi or greater, around 15 kpsi or greater, around 20 kpsi or greater, around 25 kpsi or greater, around 30 kpsi or greater, or around 35 kpsi or greater. Optionally, the step of performing mechanical lysis on mammalian producer cells comprising recombinant AAV occurs at a pressure of between around 7 kpsi and around 30 kpsi, between around 10 kpsi and around 30 kpsi, between around 15 kpsi and around 30 kpsi, between 15 kpsi and 25 kpsi, between around 20 kpsi and around 30 kpsi or around 10 kpsi or around 15 kpsi or around 20 kpsi or around 25 kpsi or around 30 kpsi.

In another embodiment of the present invention, the step of performing mechanical lysis on mammalian producer cells comprising recombinant AAV occurs at a pressure of 42.5 kpsi or lower, 40 kpsi or lower, or 37.5 kpsi or lower.

In another embodiment of the present invention, the step of performing mechanical lysis on mammalian producer cells comprising recombinant AAV occurs at a pressure of between 10 kpsi and 40 kpsi, between 15 kpsi and 40 kpsi, between 20 kpsi and 40 kpsi, between 25 kpsi and 40 kpsi, between 30 kpsi and 40 kpsi, between 35 kpsi and 40 kpsi, between 10 kpsi and 35 kpsi, between 15 kpsi and 35 kpsi, between 20 kpsi and 35 kpsi, between 25 kpsi and 35 kpsi, between 30 kpsi and 35 kpsi, between 10 kpsi and 30 kpsi, between 15 kpsi and 30 kpsi, between 15 kpsi and 25 kpsi, between 20 kpsi and 30 kpsi, or between 25 kpsi and 30 kpsi, or wherein the pressure is around 10 kpsi, around 15 kpsi, around 20 kpsi, around 25 kpsi or around 30 kpsi.

### Cell Culture

The methods or uses of the invention may comprise a step of culturing the mammalian producer cells in cell culture medium. Optionally, the mammalian producer cells are cultured under conditions suitable for rAAV production. Culturing the mammalian producer cells under conditions suitable for rAAV production refers to culturing the mammalian producer cell under conditions at which AAV can replicate. For example, the mammalian producer cell may be cultured at a temperature between 32° C. and 40° C., between 34° C. and 38° C., between 35° C. and 38° C., or around 37° C. Optionally, the mammalian producer cell may be cultured in the presence of a complete cell culture medium such as Dulbecco's Modified Eagle's Medium (DMEM). A complete cell culture medium is a medium that provides all the essential nutrients required to support the mammalian producer cell. Optionally, the complete cell culture medium is supplemented with serum, such as fetal bovine serum or bovine serum albumin. Optionally, the complete cell culture medium is serum-free.

For the purposes of the present invention, the term “suspension culture” refers to growing cells (such as mammalian producer cells) in a system suitable for suspension cell culture, i.e. a system which allows cells to grow free-floating in culture medium. Cells in a suspension system may form aggregates, or may be suspended in medium as single cells. For the purposes of the present invention, the term “adherent culture” refers to growing cells (such as mammalian producer cells) in a system suitable for adherent cell culture, i.e. for cells to be cultured whilst anchored to a substrate. Optionally, an adherent system refers to a flask or fermenter which forms a container to which cells can bind, and optionally is specifically treated to allow cell adhesion and spreading. Alternatively, an adherent system may be a “carrier system”, in which the container contains an additional carrier such as a bead or a fibre to which the cells can adhere. In such “carrier” adherent systems, the cells tend to adhere less tightly and to have a morphology that is more similar to the morphology of cells grown using suspension systems compared to cells grown in conventional adherent systems, for example, cells grown in a suspension system may have a more rounded morphology than adherent cells, which tend to be flatter.

In another embodiment, the mammalian producer cells have been cultured in suspension culture. In another embodiment, the mammalian producer cells have been cultured in adherent culture. In another embodiment, the mammalian producer cells have been cultured in adherent culture in a carrier system.

In one embodiment of the present invention, the method or use comprises a step of harvesting the mammalian producer cells. Optionally, the step of harvesting the mammalian producer cells occurs before the step of mechanical lysis. The term “harvesting the mammalian producer cells” may refer to any technique that allows the producer cells to be transferred to an apparatus suitable for carrying out the mechanical lysis step. Optionally, harvesting the mammalian producer cells may comprise concentrating them for example by centrifugation. Cells cultured in an adherent system may be harvested by detaching the cells, for example using trypsin. Harvested cells may then be resuspended before the step of mechanical lysis.

Optionally, the step of mechanical lysis occurs on mammalian producer cells comprising recombinant AAV in the cell culture medium. In other words, the mammalian producer cells are lysed in the same cell culture medium as they had been grown in, without any exchange of cell culture medium between the step of cell culture and the step of cell lysis. It is an advantage of using certain mechanical lysis techniques like microfluidisation that the mechanical lysis technique can be performed on cells suspended in cell culture medium, as this means that there is no requirement to remove the cell culture medium. If the cells are harvested before the step of mechanical lysis by, for example, centrifugation, then the cells may be resuspended in cell culture medium.

### Mammalian Producer Cells Capable of Propagating AAV

As discussed above, the mammalian producer cells are cells that are suitable for propagation of AAV. For cells to be able to propagate AAV, they should comprise genetic material encoding an AAV genome, and other genes required for AAV propagation and packaging to occur. The genetic material may be supplied, for example, by transfecting the mammalian producer cells with genetic material comprising the AAV genome and other required genes, for example in the form of plasmids. Alternatively, the mammalian producer cells may be propagated from a cell line that comprises some or all of the genetic material required, e.g. because the genetic material is comprised in the host cell genome or the genes may be supplied by infecting the mammalian producer cells with a virus. For example, the HEK293T cell line comprises an E1A gene which is an adenoviral gene that may be required for AAV propagation. Other methods of introducing genetic material, such as viral infection, may also be used.

As set out in more detail below, the genetic material required for propagating AAV may comprise rep genes (such as AAV rep genes), cap genes (such as AAV cap genes), helper genes (such as adenoviral helper genes or herpes simplex virus (HSV)-derived helper genes), and a viral genome (optionally a polynucleotide comprising two inverted terminal repeats (ITRs) and an expression cassette between the two ITRs). Optionally, the mammalian producer cells comprise sufficient genetic material for the recombinant AAV to propagate. Optionally, the mammalian producer cells comprise:
(i) a rep 52 gene;
(ii) a rep 40 gene;
(iii) a rep 68 gene;
(iv) a cap gene;
(v) a viral associated (VA) nucleic acid;
(vi) an E2a gene;
(vii) an E4 gene;
(viii) an E1A gene; and
(ix) a polynucleotide comprising an expression cassette comprising a transgene between two ITRs.

Accordingly, the methods or uses of the present invention may comprise a step of transfecting the mammalian producer cells with one or plasmids. In one embodiment of the present invention, the mammalian producer cells have been transfected with one or more plasmids. Optionally the one or more plasmids comprise an AAV cap gene and/or AAV rep genes and optionally helper genes, e.g. adenoviral helper genes or helper genes derived from herpes simplex virus (HSV), and at least one inverted terminal repeat.

Transfecting mammalian producer cells with one or more plasmids may comprise exposing the mammalian producer cells to the one or more plasmids in conditions suitable for transfection. For example, the user of the method may add a transfection agent (addition of a transfection agent would be considered to be a condition suitable for transfection), such as Polyethylenimine (PEI). Alternatively, calcium phosphate transfection, electroporation or cationic liposomes could be used. Optionally, the step of transfecting the mammalian producer cells takes place when the mammalian producer cells have grown to confluence.

Transfection may be stable or transient, i.e. cells transfected with a plasmid may stably express the genes comprised on the plasmid or may only transiently express the genes comprised on the plasmid. Furthermore, a cell may transiently express one plasmid and stably express another, e.g. a cell may be stably transfected with a plasmid comprising AAV rep and cap genes, but only transiently transfected with a plasmid comprising a transgene.

### Expression Cassette

An “expression cassette” is a nucleotide sequence comprising a transgene operably linked to a transcription regulatory element (TRE).

The term “transcription regulatory element” refers to a polynucleotide which can regulate the transcription of a gene to which it is operably linked. A TRE may comprise one or more promoter and/or enhancer elements. Suitable transcription regulatory elements include those disclosed in GB2109231.7, WO2021/084277, and WO16/181122.

The transgene may be any suitable gene. The transgene may encode a protein or a non-translated RNA which may be, for example, an siRNA or miRNA or a snRNA or an antisense RNA. The transgene may encode a protein or a non-translated RNA which is associated with a genetic disorder. The transgene may be longer than 4,000 (4 k) nucleotides, or 4,000 base pairs (4 kbp). The transgene may be longer than 4.2 k nucleotides. The transgene may be shorter than 4.4 k nucleotides.

If the preparation comprising recombinant AAV is for use in gene therapy, the transgene may be any gene that comprises or encodes a protein or nucleotide sequence that can be used to treat a disease. For example, the transgene may encode an enzyme, a metabolic protein, a signalling protein, an antibody, an antibody fragment, an antibody-like protein, an antigen, or a non-translated RNA such as a miRNA, siRNA, snRNA, or antisense RNA.

In one embodiment the transgene encodes a clotting factor, such as Factor VIII or Factor IX. The transgene may alternatively encode an enzyme, which may be a lysosomal enzyme such as alpha-galactosidase A or beta-glucocerebrosidase (GBA).

### At Least One Inverted Terminal Repeat

In one embodiment of the present invention, the one or more plasmids and/or the mammalian producer cells comprise at least one inverted terminal repeat (ITR). Thus, optionally, the one or more plasmids and/or the mammalian producer cells comprise at least one ITR, but, more typically, two ITRs (generally with one either end of the expression cassette, i.e. one at the 5′ end and one at the 3′ end). Optionally, the at least one ITR is an AAV ITR. Optionally, the at least one ITR is an AAV-derived ITR. There may be intervening sequences between the expression cassette and one or more of the ITRs. The expression cassette may be incorporated into a viral particle located between two regular ITRs or located on either side of an ITR engineered with two D regions. Optionally, the vector plasmid comprises ITR sequences which are derived from AAV1, AAV2, AAV4 and/or AAV6.

### Helper Genes

AAV can only propagate in the presence of a helper virus, which encodes proteins that aid in AAV propagation. However, growing AAV in the presence of a helper virus is not advantageous as helper viruses can be lytic to cells, including host cells used to grow AAV. Furthermore, if helper viruses are used in the production of rAAV products, such as rAAV for use in gene therapy, the helper virus may contaminate the product. As an alternative to co-infecting with helper virus such as adenovirus, the requisite genes of the helper virus can be provided in the one or more plasmids and/or the mammalian producer cells. Current understanding suggests that the following (adenoviral) helper genes are important for AAV replication: a (adenoviral) viral associated (VA) nucleic acid, an (adenoviral) E2a gene, an (adenoviral) E4 gene, and an (adenoviral) E1A gene. Accordingly, the mammalian producer cells and/or the one or more plasmids may comprise one or more of a viral associated (VA) nucleic acid, an E2a gene, an E4 gene and/or an E1A. One of more of these genes may be stably expressed in the cell type from which the mammalian producer cells are derived. For example, if the mammalian producer cells are HEK293T cells, then they will stably express the E1A gene. Suitably, the helper genes are genes corresponding to the (adenoviral) viral associated (VA) nucleic acid, the (adenoviral) E2a gene, the (adenoviral) E4 gene, and/or the (adenoviral) E1A gene from adenovirus 5. For host cells expressing the adenoviral E1A/B genes (such as HEK293T cells) the remaining required adenoviral helper genes encode E4, E2A and VA RNA I and II.

Optionally, the at least one helper virus gene is an adenovirus gene. Adenovirus is a virus which is known to aid propagation of AAV. Optionally, the at least one helper virus gene is an Adenovirus 5 gene or an Adenovirus 2 gene. The genome of Adenovirus 5 is set out in SEQ ID NO: 2, and the genome of Adenovirus 2 with Genbank Sequence ID AC_000007.1 is set out in SEQ ID NO: 3. Accordingly, the helper genes may comprise a stretch of nucleotides present in SEQ ID NO: 2 or SEQ ID NO: 3, or a corresponding stretch of nucleotides in another serotype of adenovirus.

The helper genes of adenoviruses encode E1A, E1B, E4, E2A and VA RNA I and II.

E1A is encoded by nucleotides 560-1545 of the Adenovirus 5 genome (for example the genome of SEQ ID NO: 2). Nucleotides 560-1545 contain an intron, from nucleotide 1113 to nucleotide 1228. This intron is not essential, and so an E1A gene comprising nucleotides 560-1112 and 1229-1545 of SEQ ID NO: 2 would encode a functional E1A protein.

E1B is actually two proteins E1B 19K and E1B 55K, which work together to block apoptosis in adenovirus-infected cells. E1B is encoded by nucleotides 1714-2244 (E1B 19K), and by nucleotides 2019-3509 (E1B 55K) of the Adenovirus 5 genome (for example the genome of SEQ ID NO: 2).

E4 is encoded by a number of different open reading frames (ORFs) of the Adenovirus 5 genome (for example the genome of SEQ ID NO: 2). E4 ORF 6/7 is encoded by nucleotides 32914-34077, which comprises an intron between nucleotides 33193 and 33903. This intron is not essential, and so an E4 ORF 6/7 comprising nucleotides 32914-33192 and 33904-34077 of SEQ ID NO: 2 is sufficient. E4 34K is encoded by nucleotides 33193-34077 of the Adenovirus 5 genome (for example the genome of SEQ ID NO: 2). E4 ORF 4 is encoded by nucleotides 33998-34342 of the Adenovirus 5 genome (for example the genome of SEQ ID NO: 2). E4 ORF 3 is encoded by nucleotides 34353-34703 of the Adenovirus 5 genome (for example the genome of SEQ ID NO: 2). E4 ORF 2 is encoded by nucleotides 34700 to 35092 of the Adenovirus 5 genome (for example the genome of SEQ ID NO: 2). E4 ORF 1 is encoded by nucleotides 35140-35526 of the Adenovirus 5 genome (for example the genome of SEQ ID NO: 2).

A functional E4 protein may only comprise amino acids encoded by ORFs 6 and 7, as only the amino acids encoded by ORFs 6 and 7 are required for activity. Optionally, therefore, the functional E4 protein comprises a polypeptide sequence encoded by all or a significant portion of ORFs 6 and 7. Optionally, the functional E4 protein does not comprise polypeptide sequence encoded by all or a portion of ORFs 1-4 and 34K. However, the amino acids encoded by ORFs 1-3 and 34K do improve the activity of the E4 protein, and so in some embodiments the functional E4 protein comprises amino acids encoded by ORFs 1-7.

The E2 (E2A) gene is encoded by nucleotides 22443-24032 of the Adenovirus 5 genome (for example the genome of SEQ ID NO: 2).

The VA RNA I and II is encoded by nucleotides 10589-11044 of the Adenovirus 5 genome (for example the genome of SEQ ID NO: 2).

### Rep Genes

The one or more plasmids and/or mammalian producer cells may comprise AAV rep genes. The mammalian producer cells may comprise (i) a rep 52 gene, (ii) a rep 40 gene, and/or (iii) a rep 68 gene. AAV comprises a rep gene region which encodes four Rep proteins (Rep 78, Rep 68, Rep 52 and Rep 40). The rep gene region is under the control of the p5 and p19 promoters. When the p5 promoter is used, a gene that encodes Rep 78 and Rep 68 is transcribed. Rep 78 and Rep 68 are two alternative splice variants (Rep 78 comprises an intron that is excised in Rep 68). Similarly, when the p19 promoter is used, a gene that encodes Rep 52 and Rep 40 is transcribed. Rep 52 and Rep 40 are alternative splice variants (Rep 52 comprises an intron that is excised in Rep 40).

The four Rep proteins are known to be involved in replication and packaging of the viral genome, and are, therefore, useful in rAAV production.

It is not necessary for all four Rep proteins to be present. Optionally, however, the at least one rep gene encodes a large Rep protein (Rep 78 or Rep 68) and a small Rep protein (Rep 52 or Rep 40). Accordingly, the one or more plasmids and/or the mammalian producer cells may comprise at least one rep gene encoding:
(a) a functional (AAV) Rep 52 protein, i.e. a rep 52 gene;
(b) a functional (AAV) Rep 40 protein, i.e. a rep 40 gene; and/or
(c) a functional (AAV) Rep 68 protein, i.e. a rep 68 gene.

For example, the one or more plasmids and/or the mammalian producer cells may comprise a (AAV) rep 52 gene, a (AAV) rep 40 gene, and/or a (AAV) rep 68 gene. Optionally, the one or more plasmids and/or the mammalian producer cells comprises a (AAV) rep 78 gene.

A “functional” Rep protein is one which allows for production of AAV particles. In particular, Rep 78 or Rep 68 (the large Rep proteins) are believed to be involved in replication of the AAV genome, and Rep 52 and Rep 40 (the small Rep proteins) are believed to be involved in packaging of the AAV genome into a capsid. It is within the abilities of the skilled person to determine whether a given Rep protein is functional. The skilled person merely needs to determine whether the Rep protein supports AAV production using an AAV production assay for example using a “test” mammalian producer cell which is identical to the “reference” mammalian producer cell except that it comprises a gene encoding the Rep protein whose function is to be tested in place of the reference rep gene.

In general, a Rep protein will only be able to support rAAV production if it is compatible with the ITR(s) surrounding the genome of the AAV to be packaged. Some Rep proteins may only be able to package genomic material (such as an expression cassette) when it is flanked by ITR(s) of the same serotype as the Rep protein. Other Rep proteins are cross-compatible, meaning that they can package genomic material that is flanked by ITR(s) of a different serotype. For example, it is preferred that the Rep protein is able to support replication and packaging of an expression cassette comprised within the one or more plasmids, and such a Rep protein will be compatible with the at least one ITR flanking the expression cassette (i.e. able to replicate and package the expression cassette flanked on at least one side by an ITR).

Optionally, the one or more plasmids and/or the mammalian producer cells comprise a gene encoding a functional (AAV) Rep 52 protein (a rep 52 gene), at least one gene encoding a functional (AAV) Rep 40 protein (a rep 40 gene), and a gene encoding a functional (AAV) Rep 68 protein (a rep 68 gene).

The one or more plasmids and/or mammalian producer cells may comprise two genes encoding a functional (AAV) Rep 40 protein. For example, the one or more plasmids and/or mammalian producer cells may comprise two rep genes that are separated on the plasmid.

SEQ ID NO: 1 provides the sequence of the genome of wild type AAV2, and nucleotides 321-2252 of SEQ ID NO: 1 encode the four Rep proteins. The full-length rep gene (nucleotides 321-2252) encodes all four Rep proteins (Rep 78 and Rep 68 from the p5 promoter and Rep 52 and Rep 40 from the p19 promoter). A shorter stretch of the rep gene downstream of the p19 promoter (nucleotides 993-2252) encodes Rep 52 and Rep 40 only (i.e. this stretch of the rep gene reaches from the end of the p19 promoter to the end of the gene). Nucleotides 1907-2227 of SEQ ID NO: 1 correspond to an intron. Rep 78 and Rep 52 comprise amino acids encoded by the intron, but Rep 68 and Rep 40 are alternative splice variants that do not comprise amino acids encoded by the intron.

Optionally, the one or more plasmids and/or the mammalian producer cells comprise a gene encoding a functional Rep 52 protein (a rep 52 gene), and the rep 52 gene comprises a nucleic acid sequence having at least 95%, at least 98%, at least 99%, or 100% identity to the full length or a fragment of at least 800, at least 900, at least 1000, or at least 1100 nucleotides in length of nucleotides 993-2186 of SEQ ID NO: 1, or to a corresponding stretch of nucleotides in a different serotype of AAV.

It is within the abilities of the person skilled in the art to determine whether a particular (test) stretch of nucleotides is a “corresponding stretch of nucleotides in a different serotype of” AAV. All that is required is that the person skilled in the art align the test stretch of nucleotides with the genome of the reference serotype (i.e. SEQ ID NO: 1). If the test stretch of nucleotides has greater than 90% identity with a contiguous stretch of nucleotides of the same length in SEQ ID NO: 1, the contiguous stretch is a corresponding stretch of nucleotides in a different serotype of AAV. The same applies in the case of adenovirus sequences (except here the reference serotype is SEQ ID NO: 2).

Optionally, the one or more plasmids and/or the mammalian producer cells comprise a gene encoding a functional Rep 40 protein (a rep 40 gene), and the rep 40 gene comprises a nucleic acid sequence having at least 95%, at least 98%, at least 99%, or 100% identity to the full length or to a fragment of at least 600, at least 700, at least 800, or at least 900 nucleotides in length of a stretch of nucleotides corresponding to nucleotides 993-2252 minus nucleotides 1907-2227 of SEQ ID NO: 1, or to corresponding stretches of nucleotides in a different serotype of AAV. Hence, such a rep 40 gene has at least the above-specified identity to a notional stretch of nucleotides consisting of nucleotides 993-1906 of SEQ ID NO: 1 immediately juxtaposed with nucleotides 2228-2252 of SEQ ID NO: 1 (5′-[993-1906]-[2228-2252]-3′) or to a notional stretch of nucleotides from a different AAV serotype.

Optionally, the one or more plasmids and/or the mammalian producer cells comprises at least one gene encoding a functional Rep 40 protein (i.e. a rep 40 gene), and the rep 40 gene comprises a nucleic acid sequence having at least 95%, at least 98%, at least 99%, or 100% identity to the full-length or to a fragment of at least 900, at least 1000, at least 1100, or at least 1200 nucleotides in length of nucleotides 993-2252 of SEQ ID NO: 1, or to a corresponding stretch of nucleotides in a different serotype of AAV.

Optionally, the one or more plasmids and/or the mammalian producer cells comprise a gene encoding a functional Rep 68 protein (i.e. a rep 68 gene), and the rep 68 gene comprises a nucleic acid sequence having at least 95%, at least 98%, at least 99%, or 100% identity to the full-length or to a fragment of at least 1000, at least 1400, at least 1500, or at least 1600 nucleotides in length of a stretch of nucleotides corresponding to nucleotides 321-2252 minus nucleotides 1907-2227 of SEQ ID NO: 1, or to corresponding stretches of nucleotides in a different serotype of AAV. Hence, such a rep 68 gene has at least the above-specified identity to a notional stretch of nucleotides consisting of nucleotides 321-1906 of SEQ ID NO: 1 immediately juxtaposed with nucleotides 2228-2252 of SEQ ID NO: 1 (5′-[321-1906]-[2228-2252]-3′), or to a notional stretch of nucleotides from a different AAV serotype.

Optionally, the one or more plasmids and/or mammalian producer cells comprise a gene encoding functional Rep 68 and Rep 40 proteins (i.e. a rep 68 gene and a rep 40 gene), wherein said gene comprises a nucleic acid having at least 95%, at least 98%, at least 99%, or 100% identity to the full length or to a fragment of at least 1400, 1500, 1600 or 1700 nucleotides in length of the following stretches of native AAV2 sequence (SEQ ID NO: 1) positioned in immediate juxtaposition from 5′ to 3′: 200-1906; 2228-2309, or to corresponding juxtaposed stretches of nucleotides from a different serotype of AAV.

Optionally, the one or more plasmids and/or the mammalian producer cells comprise a gene encoding functional Rep 52 and Rep 40 proteins, wherein said gene comprises a nucleic acid having at least 95%, at least 98%, at least 99%, or 100% identity to the full length or to a fragment of at least 1300, 1400, 1500 or 1600 nucleotides in length of the following stretch of native AAV2 sequence (SEQ ID NO: 1): 658-2300, or to a corresponding stretch of nucleotides from a different serotype of AAV.

Optionally, the one or more plasmids and/or the mammalian producer cells comprise a stretch of nucleotides encoding functional Rep 68, Rep 52 and Rep 40 proteins, wherein said stretch comprises a nucleic acid having at least 95%, at least 98%, at least 99%, or 100% identity to the full length or to a fragment of at least 3000, 3200, 3300 or 3400 nucleotides in length of the following stretches of native AAV2 sequence (SEQ ID NO: 1) positioned in immediate juxtaposition from 5′ to 3′: 200-1906; 2228-2309; 658-2300, or to corresponding juxtaposed stretches of nucleotides from a different serotype of AAV.

Optionally, the one or more plasmids and/or the mammalian producer cells do not comprise a contiguous sequence of at least 1700, at least 1800, or 1866 nucleotides corresponding to a contiguous stretch of nucleotides of equivalent length comprised within nucleotides 321-2186 of SEQ ID NO: 1, or within a corresponding stretch of nucleotides in a different serotype of AAV. The contiguous stretch of nucleotides comprised within nucleotides 321-2186 corresponds to Rep 78.

### Cap Gene

The one or more plasmids and/or the mammalian producer cells may comprise a (AAV) cap gene. The cap gene encodes a functional Cap protein. The cap gene may encode a functional set of Cap proteins. AAV generally comprise three Cap proteins, VP1, VP2 and VP3. These three proteins form a capsid into which the AAV genome is inserted, and allow the transfer of the AAV genome into a host cell. All of VP1, VP2 and VP3 are encoded in native AAV by a single gene, the cap gene. The amino acid sequence of VP1 comprises the sequence of VP2. The portion of VP1 which does not form part of VP2 is referred to as VP1 unique or VP1U. The amino acid sequence of VP2 comprises the sequence of VP3. The portion of VP2 which does not form part of VP3 is referred to as VP2unique or VP2U.

A “functional” set of Cap proteins is one which allows for encapsidation of AAV. It is within the abilities of the skilled person to determine whether a given Cap protein is or a set of Cap proteins are functional. The skilled person merely needs to determine whether the encoded Cap protein(s) support AAV production using an AAV production assay. The Cap protein(s) will be considered to be “functional” if it/they support(s) rAAV production at a level at least 25%, at least 40%, at least 50%, at least 70%, at least 80%, at least 90% or at least 95% of the level supported by the wild type cap gene product. Preferably, the Cap protein(s) will be considered to be “functional” if it/they support(s) rAAV production at a level at least 70%, at least 80%, at least 90% or at least 95% of the level supported by the wild-type cap gene product.

Optionally, VP2 and/or VP3 proteins are “functional” if an AAV comprising the VP2 and/or the VP3 proteins is able to transduce mammalian cells susceptible to infection with the AAV, e.g. Huh7 cells at a level at least 25%, at least 40%, at least 50%, at least 70%, at least 80%, at least 90% or at least 95% of that of an equivalent AAV comprising a wild type VP2 and/or VP3 protein. The ability of an AAV particle to transduce mammalian host cells, e.g. Huh7 cells can be tested by adding a reporter protein such as green fluorescent protein (GFP) to the AAV particle, mixing the AAV particle with Huh7 cells, and measuring the fluorescence produced.

Optionally, the one or more plasmids and/or the mammalian producer cells comprise a cap gene that encodes a VP1, a VP2 and/or a VP3 protein. Optionally, the VP1, VP2 and VP3 proteins are expressed from more than one cap gene. Optionally, the one or more plasmids and/or the mammalian producer cells comprise a cap gene that encodes a VP1, a VP2 and a VP3 protein. Optionally the one or more plasmids and/or the mammalian producer cells comprise a cap gene encoding a functional VP1, i.e. a VP1 protein capable of assembling with other Cap proteins to encapsidate a viral genome.

Different serotypes of AAV have Cap proteins having different amino acid sequences. A cap gene encoding any (set of) Cap protein(s) is suitable for use in connection with the present invention. The Cap protein can be a native Cap protein expressed in AAV of a certain serotype. Alternatively, the Cap protein can be a non-natural, for example an engineered, Cap protein, which is designed to comprise a sequence different to that of a native AAV Cap protein. Genes encoding non-natural Cap proteins are particularly advantageous, as in the context of gene therapy applications it is possible that fewer potential patients have levels of antibodies that prevent transduction by AAV comprising non-natural Cap proteins, relative to native capsids.

Optionally, the cap gene encodes a Cap protein from a serotype selected from the group consisting of serotypes 1, 2, 3A, 3B, 4, 5, 6, 7, 8, 9, 10, 11, 12, or 13. Optionally, the cap gene encodes a Cap protein selected from the group consisting of LK03, rh74, rh10 and Mut C (WO 2016/181123; WO 2013/029030; WO 2017/096164).

Optionally, the cap gene encodes a Cap protein from a serotype selected from serotypes 1, 2, 3A, 3B, 4, 5, 6, 7, 8, 9, 10, 11, 12, and 13. Optionally, the cap gene encodes a Cap protein selected from LK03, rh74, rh10 and Mut C (WO 2016/181123; WO 2013/029030; WO 2017/096164).

### Sufficient Genetic Material

“Sufficient genetic material” refers to the minimum required genetic material for a mammalian producer cell to produce rAAV, or for AAV to propagate in the mammalian producer cells, when cultured under conditions suitable for rAAV production. Sufficient genetic material may comprise:
(i) a rep 52 gene;
(ii) a rep 40 gene;
(iii) a rep 68 gene;
(iv) a cap gene;
(v) a VA nucleic acid;
(vi) an E2a gene;
(vii) an E4 gene;
(viii) an E1A gene; and
(ix) a polynucleotide comprising an expression cassette comprising a transgene between the two ITRs.

The skilled person can easily determine if a mammalian producer cell comprises sufficient genetic material for the production of rAAV by culturing the mammalian producer cell under conditions suitable for the production of rAAV, e.g. as described under the heading Cell culture, and assaying for the presence of rAAV, for example by detecting the viral genome titre using qPCR, for example, as described under the heading Viral genome titre assay or detecting the capsid titre using an ELISA, for example, as described under the heading Capsid titre assay. Alternatively, a suitable AAV production assay is described under the heading AAV production assay. A mammalian producer cell comprises sufficient genetic material for the production of rAAV if rAAV is detected in an AAV production assay after the mammalian producer cell has been cultured under conditions suitable for the production of rAAV.

The genetic material may be comprised in one or more plasmids. The genetic material may be comprised within the genome of the mammalian producer cell. For example, the genome of the mammalian producer cell may comprise an E1A gene.

In one embodiment of the present invention, the mammalian producer cells have been transfected with one or more plasmids comprising an AAV cap gene and/or AAV rep genes, and optionally helper genes, e.g. adenoviral helper genes or helper genes derived from herpes simplex virus (HSV), and at least one inverted terminal repeat (ITR).

In another embodiment, the method or use further comprises a step of transfecting the mammalian producer cells with one or more plasmids comprising an AAV cap gene and/or AAV rep genes, and optionally helper genes, e.g. adenoviral helper genes or helper genes derived from herpes simplex virus (HSV), and at least one inverted terminal repeat (ITR).

Suitably, the genetic material (i.e. the sufficient genetic material for the production of rAAV) may be conferred by infection of the mammalian producer cells with a suitable helper virus. Suitable helper viruses include herpesviruses, e.g. HSV-1, and adenovirus, e.g. adenovirus 2 or adenovirus 5. In one embodiment of the present invention, the mammalian producer cells have been infected with one or more helper virus comprising an AAV cap gene and/or AAV rep genes, and/or optionally helper genes, e.g. adenoviral helper genes, and/or at least one inverted terminal repeat (ITR).

In another embodiment, the method or use further comprises a step of infecting the mammalian producer cells with one or more helper virus comprising an AAV cap gene and/or AAV rep genes, and/or optionally helper genes, e.g. adenoviral helper genes, and/or at least one inverted terminal repeat (ITR).

The skilled person is also aware of other methods to introduce sufficient genetic material for the production of rAAV into the mammalian producer cells, e.g. by using synthetic DNA, e.g. dbDNA™ or hpDNA™.

### At a Temperature of . . .

The methods and uses comprising a step of mechanical lysis according to the present invention may be performed at different temperatures. It is advantageous to keep the temperature at which the mechanical lysis is performed low in order to minimise denaturation of the rAAV proteins and genome. A microfluidisation machine may comprise a cooling unit to reduce the temperature of a sample as it undergoes lysis.

In one aspect of the present invention, the step of mechanical lysis, suitably microfluidisation, occurs at a temperature of 15° C. or lower, 10° C. or lower, 8° C. or lower, 5° C. or lower, between 5° C. and 10° C., or between 0° C. and 5° C. A step of mechanical lysis occurs at a temperature of around 15° C. or lower if a mechanical lysis apparatus such as a microfluidisation machine is set at a temperature of 15° C. or lower. In one embodiment, the step of mechanical lysis, suitably microfluidisation, occurs at a temperature of around 10° C. or lower. In one embodiment, the step of mechanical lysis, suitably microfluidisation, occurs at a temperature of around 5° C. or lower.

### Increased Viral Genome Titre

The methods and uses of the invention may result in a preparation that comprises recombinant AAV at an increased or high viral genome titre. Viral genome titre is the concentration of viral genome particles present in a preparation. If the preparation comprises rAAV, the viral genome particles will be AAV viral genome particles. Viral genome titre can be used as a measure of the yield of rAAV. A preparation comprising recombinant AAV produced by the methods or uses of the invention may have increased viral genome titre when compared to a preparation comprising recombinant AAV produced by a corresponding method or use comprising a step of performing mechanical lysis at a pressure of 5 kpsi. Suitably, a preparation comprising recombinant AAV produced by the methods or uses of the invention may have increased viral genome titre when compared to a preparation comprising recombinant AAV produced by a corresponding method or use comprising a step of performing mechanical lysis at a pressure of 10 kpsi. In one embodiment, the method or use according to the invention produces a preparation comprising recombinant AAV having increased viral genome titre. The preparation comprising recombinant AAV is the preparation that is produced by the methods and uses of the invention. Accordingly, to determine whether a method or use produces a preparation comprising recombinant AAV having increased viral genome titre, the viral genome titre of the preparation should be measured once the method or use has been completed, i.e. after the step of performing mechanical lysis and/or if the method or use comprises further subsequent steps, the viral genome titre of the preparation should be measured once those further subsequent steps have been completed, and compared to the viral genome titre of a preparation made using an equivalent method or use comprising a step of performing mechanical lysis at a pressure of 5 kpsi or at a pressure of 10 kpsi and/or lacking a step of performing mechanical lysis.

In another embodiment, the method or use according to the invention produces a preparation comprising recombinant AAV having at least 10%, at least 20%, at least 30%, at least 40%, at least 50%, or at least 60% increased viral genome titre when compared to a preparation comprising recombinant AAV produced by a corresponding method or use comprising a step of performing mechanical lysis at a pressure of 5 kpsi.

In another embodiment, the method or use according to the invention produces a preparation comprising recombinant AAV having at least 10%, at least 20%, at least 30%, at least 40%, at least 50%, or at least 60% increased viral genome titre when compared to a preparation comprising recombinant AAV produced by a corresponding method or use comprising a step of performing mechanical lysis at a pressure of 10 kpsi.

In another embodiment, the method or use according to the invention is a method or use for increasing the viral genome titre of a preparation comprising recombinant AAV.

In another embodiment, the method or use according to the invention is a method or use for increasing the viral genome titre of a preparation comprising recombinant AAV by at least 10%, at least 20%, at least 30%, at least 40%, at least 50%, or at least 60%, when compared to a preparation comprising recombinant AAV produced by a corresponding method or use comprising a step of performing mechanical lysis at a pressure of 5 kpsi.

In another embodiment, the method or use according to the invention is a method or use for increasing the viral genome titre of a preparation comprising recombinant AAV by at least 10%, at least 20%, at least 30%, at least 40%, at least 50%, or at least 60%, when compared to a preparation comprising recombinant AAV produced by a corresponding method or use comprising a step of performing mechanical lysis at a pressure of 10 kpsi.

In another embodiment, the step of performing mechanical lysis according to the invention increases the viral genome titre of the preparation comprising recombinant AAV.

In another embodiment, the step of performing mechanical lysis according to the invention increases the viral genome titre of the preparation comprising recombinant AAV by at least 10%, at least 20%, at least 30%, at least 40%, at least 50%, or at least 60%, when compared to a preparation comprising recombinant AAV produced by a corresponding method or use comprising a step of performing mechanical lysis at a pressure of 5 kpsi. In another embodiment, the step of performing mechanical lysis according to the invention increases the viral genome titre of the preparation comprising recombinant AAV by at least 10%, at least 20%, at least 30%, at least 40%, at least 50%, or at least 60%, when compared to a preparation comprising recombinant AAV produced by a corresponding method or use comprising a step of performing mechanical lysis at a pressure of 10 kpsi. To determine whether a step of performing mechanical lysis increases the viral genome titre of the preparation, the viral genome titre of the preparation should be measured immediately after the step is performed, i.e., before any steps subsequent to the step of performing mechanical lysis. The viral genome titre should be compared to the viral genome titre measured immediately after a step of performing mechanical lysis and/or at an equivalent point in an equivalent method or use lacking a step of performing mechanical lysis. For example, to determine if a step of performing microfluidisation at a pressure of 7 kpsi or greater has increased the viral genome titre of a preparation comprising recombinant AAV, the viral genome titre should be measured immediately after the step of microfluidisation and compared to the viral genome titre measured immediately after a step of performing mechanical lysis at a pressure of 5 kpsi and/or at an equivalent point in an equivalent method or use lacking a step of performing microfluidisation at a pressure of 7 kpsi or greater.

### Viral Genome Titre Assay

The skilled person will understand that there are many suitable methods for determining the viral genome titre of a preparation comprising recombinant AAV. Viral genome titre may be measured using digital droplet polymerase chain reaction (ddPCR). Viral genome titre may be measured by the quantitative polymerase chain reaction (qPCR). qPCR or ddPCR may be carried out with primers specific to the viral genome. For example, if the viral genome is the AAV genome, primers specific to the AAV genome will be used. A suitable qPCR viral genome assay is described in the Examples.

The AAV viral genome assay may be based on a quantitative polymerase chain reaction (qPCR) specific for the promoter sequence of the rAAV expression cassette. In principle, the qPCR primers can be designed to bind any part of the recombinant AAV genome which is not common to wild type AAV genomes, but it is recommended against using primer template sequences very close to the ITRs as doing so can lead to an exaggerated vector genome titre measurement. Suitably, qPCR is carried out using a pair of primers that are able to amplify at least a region of the promoter of the expression cassette. Optionally, at least one of the primers is specific for (reverse and complementary to or identical to depending on whether the primer is a forward primer or a reverse primer) a region of at least 12, at least 14, at least 16, or at least 18 nucleotides of the promoter of the expression cassette. Optionally, one primer is specific for the start of the promoter (the first at least 12 nucleotides of the promoter) and the other primer is specific for a region of the expression cassette that is about 150 base pairs from the binding site of the first primer. The qPCR may be performed using SYBR green or another intercalating dye that can be used for detection of the amplification product. Alternatively, the qPCR product may be detected using Taqman® assay or similar. An exemplary qPCR assay for determination of the viral genome titre is described in the Examples below.

Cell lysate test samples may be subjected to a nuclease treatment procedure in order to remove non-packaged vector genomes prior to performing qPCR or ddPCR.

“Droplet digital PCR” (ddPCR) refers to a digital PCR assay that measures absolute quantities by counting nucleic acid molecules encapsulated in discrete, volumetrically defined, water-in-oil droplet partitions that support PCR amplification (i.e., a plurality of such compartments). Typically, a “droplet” refers to water-in-oil droplet (i.e., an oil droplet that may be generated by emulsifying a sample with droplet generator oil); an individual partition of the droplet digital PCR assay. A droplet supports PCR amplification of template molecule(s) using homogenous assay chemistries and workflows similar to those widely used for real-time PCR applications (Hinson et al (2011) Anal. Chem. 83:8604-8610; Pinheiro et al (2012) Anal. Chem. 84:1003-1011). A single ddPCR reaction may typically be comprised of at least 5000, at least 6000, at least 7000, at least 8000, at least 9000, at least 10,000, at least 12,000, at least 14,000, at least 16,000, at least 18,000 or at least 20,000 compartments.

Droplet digital PCR may be performed using any platform that performs a digital PCR assay that measures absolute quantities by counting nucleic acid molecules encapsulated in discrete, volumetrically defined, water-in-oil droplet partitions that support PCR amplification. The strategy for droplet digital PCR may be summarized as follows: a sample is diluted and partitioned into thousands to millions of separate reaction chambers (water-in-oil droplets) so that each contains one or no copies of the nucleic acid molecule of interest. The number of positive droplets detected, which contain the target amplicon (i.e., nucleic acid molecule of interest), versus the number of negative droplets, which do not contain the target amplicon (i.e., nucleic acid molecule of interest), may be used to determine the number of copies of the nucleic acid molecule of interest that were in the original sample. Examples of droplet digital PCR systems include the QX100™ Droplet Digital PCR System by Bio-Rad, which partitions samples containing nucleic acid template into about 20,000 nanoliter-sized droplets.

Droplet digital PCR may thus be used to detect a single target in a sample, for example using a single primer pair. However, ddPCR may also be used to detect two different targets in a sample, for example using two primer pairs, each primer pair hybridising to a different target, i.e., duplexing of targets. Duplexing may be extended to look at more targets in a sample, such as at least 3, at least 4, at least 5, at least 6, at least 7, at least 8, at least 9, at least 10, at least 11 or at least 12 different targets in a sample, i.e., multiplexing of targets. Duplexing and multiplexing with ddPCR allows for improved sensitivity and precision, increased low level detection, and also the inference of the size of a nucleic acid. ddPCR may be quantitative.

### Increased Capsid Titre

Capsid titre is the concentration of capsid particles present in a preparation. For example, if the preparation comprises rAAV, the capsid titre is the concentration of AAV capsid particles in the preparation. The preparation comprising recombinant AAV may have increased capsid titre when compared to a preparation comprising recombinant AAV produced by a corresponding method or use comprising a step of performing mechanical lysis at a pressure of 5 kpsi. Suitably, the preparation comprising recombinant AAV may have increased capsid titre when compared to a preparation comprising recombinant AAV produced by a corresponding method or use comprising a step of performing mechanical lysis at a pressure of 10 kpsi.

In one embodiment, the method or use according to the invention produces a preparation comprising recombinant AAV having increased capsid titre. The preparation comprising recombinant AAV is the preparation that is produced by the methods and uses of the invention. Accordingly, to determine whether a method or use produces a preparation comprising recombinant AAV increased capsid titre, the capsid titre of the preparation should be measured once the method or use has been completed, i.e. after the step of performing mechanical lysis and if the method or use comprises further subsequent steps once those further subsequent steps have been completed, and compared to the capsid titre of a preparation made using an equivalent method or use comprising a step of performing mechanical lysis and/or lacking a step of performing mechanical lysis.

In another embodiment, the method or use according to the invention produces a preparation comprising recombinant AAV having at least 10%, at least 20%, at least 30%, at least 40%, at least 50%, at least 60%, at least 70%, or at least 80% increased capsid titre when compared to a preparation comprising recombinant AAV produced by a corresponding method or use comprising a step of performing mechanical lysis at a pressure of 5 kpsi.

In another embodiment, the method or use according to the invention produces a preparation comprising recombinant AAV having at least 10%, at least 20%, at least 30%, at least 40%, at least 50%, at least 60%, at least 70%, or at least 80% increased capsid titre when compared to a preparation comprising recombinant AAV produced by a corresponding method or use comprising a step of performing mechanical lysis at a pressure of 10 kpsi.

In another embodiment, the method or use according to the invention is a method or use for increasing the capsid titre of a preparation comprising recombinant AAV.

In another embodiment, the method or use according to the invention is a method or use for increasing the capsid titre of a preparation comprising recombinant AAV by at least 10%, at least 20%, at least 30%, at least 40%, at least 50%, at least 60%, at least 70%, or at least 80%, when compared to a preparation comprising recombinant AAV produced by a corresponding method or use comprising a step of performing mechanical lysis at a pressure of 5 kpsi.

In another embodiment, the method or use according to the invention is a method or use for increasing the capsid titre of a preparation comprising recombinant AAV by at least 10%, at least 20%, at least 30%, at least 40%, at least 50%, at least 60%, at least 70%, or at least 80%, when compared to a preparation comprising recombinant AAV produced by a corresponding method or use comprising a step of performing mechanical lysis at a pressure of 10 kpsi.

In another embodiment, the step of performing mechanical lysis according to the invention increases the capsid titre of the preparation comprising recombinant AAV.

In another embodiment, the step of performing mechanical lysis according to the invention increases the capsid titre of the preparation comprising recombinant AAV by at least 10%, at least 20%, at least 30%, at least 40%, at least 50%, at least 60%, at least 70%, or at least 80%, when compared to a preparation comprising recombinant AAV produced by a corresponding method or use comprising a step of performing mechanical lysis at a pressure of 5 kpsi. In another embodiment, the step of performing mechanical lysis according to the invention increases the capsid titre of the preparation comprising recombinant AAV by at least 10%, at least 20%, at least 30%, at least 40%, at least 50%, at least 60%, at least 70%, or at least 80%, when compared to a preparation comprising recombinant AAV produced by a corresponding method or use comprising a step of performing mechanical lysis at a pressure of 10 kpsi. To determine whether a step of performing mechanical lysis increases the viral genome titre of the preparation, the viral genome titre of the preparation should be measured immediately after the step is performed, i.e., before any steps subsequent to the step of performing mechanical lysis. The viral genome titre should be compared to the viral genome titre measured immediately after a step of performing mechanical lysis and/or at an equivalent point in an equivalent method or use lacking a step of performing mechanical lysis. For example, to determine if a step of performing microfluidisation at a pressure of 7 kpsi or greater has increased the capsid titre of a preparation comprising recombinant AAV, the capsid titre should be measured immediately after the step of microfluidisation and compared to the capsid titre measured immediately after a step of performing mechanical lysis at a pressure of 5 kpsi and/or at an equivalent point in an equivalent method or use lacking a step of performing microfluidisation at a pressure of 7 kpsi or greater.

### Capsid Titre Assay

The skilled person will understand that there are many suitable methods for determining the capsid titre of a preparation comprising recombinant AAV. Capsid titre may be measured by an enzyme-linked immunosorbent assay (ELISA). For example, the capsid-specific ELISA may comprise exposing the rAAV preparation to an antibody that binds to the capsid protein. If, for example, the vector plasmid comprises a cap gene that encodes a capsid from an AAV2 serotype, the antibody may be an antibody that binds to the AAV2 capsid. For example, the user may coat a plate with an antibody that is specific for the capsid. The user may then pass the rAAV preparation over the surface of the plate. The capsids will bind to the antibody and be immobilised on the plate. The plate may then be washed to remove contaminants. The amount of capsids present can then be detected by addition of a detection antibody that can bind to the capsid and is conjugated to a detection agent such as streptavidin peroxidase. The amount of capsids present will be proportional to the colour change obtained when the streptavidin peroxidase is exposed to the chromogenic substrate TMB (tetramethylbenzidine). A suitable capsid titre assay is described in the Example.

In one aspect of the present invention, the capsid titre is measured by ELISA.

### Endonuclease Treatment

An endonuclease is an enzyme capable of breaking down polynucleotides in a preparation. Endonuclease treatment may be used to remove unpackaged viral genomes or host cell DNA impurities. Endonuclease treatment therefore improves the purity of a preparation comprising rAAV. The skilled person would understand that there are many suitable endonuclease enzymes which could be used in a step of endonuclease treatment. Suitably, the endonuclease enzyme is Denarase®. Suitably, the endonuclease enzyme is Benzonase®. Suitably, the endonuclease enzyme is Turbonuclease™. The skilled person would understand that there are many suitable methods of performing endonuclease treatment. A suitable method is set out in the Examples.

Optionally, a preparation comprising recombinant AAV may be treated with endonuclease at 20 units/ml (U/ml). Optionally, the treatment comprises the step of incubating the preparation comprising recombinant AAV with the endonuclease. Optionally, the step of incubation lasts for around 1 hour, around 2 hours, around 3 hours, around 4 hours, around 5 hours, around 6 hours, around 7 hours, around 8 hours, around 9 hours, around 10 hours, around 11 hours, around 12 hours, around 13 hours, around 14 hours, around 15 hours, around 16 hours, around 17 hours, around 18 hours, around 19 hours, around 20 hours, around 21 hours, around 22 hours, around 23 hours, around 24 hours or overnight. Suitably, the endonuclease incubation step is performed at room temperature, e.g. a temperature of around 18° C. to around 25° C.

The methods or uses according to the present invention may further comprise a step of endonuclease treatment. Optionally, the step of endonuclease treatment occurs after the step of mechanical lysis.

### Depth Filtration

The methods or uses according to the present invention may further comprise a step of depth filtration. Depth filtration comprises passing a preparation through a depth filter at a particular flux in order to remove impurities such as host cell debris. Removing impurities may increase the concentration of rAAV in a preparation, and thus increase the viral genome and capsid titres. In addition to removing impurities depth filtration of the preparation will improve the purity/quality of the preparation. The examples also demonstrate that adding a step of depth filtration may improve the viral genome titre and/or viral genome/capsid ratio of the preparation.

The skilled person would understand that there are many commercially available depth filters. Depth filters have a micron rating, which describes the range of particle sizes which may pass through the depth filter and will therefore be retained in the preparation after performing the step of depth filtration. For example, a depth filter may have a micron rating of 0.2-1.1 μm, meaning that particles of a size between 0.2 and 1.1 μm will pass through the filter, and larger particles will not. Depth filters may comprise an organic filter and an inorganic filter aid. For example, the organic filter may comprise cellulose fibres, and the inorganic filter aid may comprise a perlite or resin or diatomaceous earth. A suitable depth filter is a C0HC filter. Suitably, the depth filtration step is performed at room temperature, e.g. a temperature of around 18° C. to around 25° C.

In one embodiment, the step of depth filtration occurs after the step of mechanical lysis. In another embodiment, the step of depth filtration occurs after the step of endonuclease treatment.

The “flux” at which depth filtration occurs is a measure of the speed at which the preparation passes through the depth filter. Higher flux means that the preparation is passing through the depth filter at an increased rate. The skilled person can easily determine the flux at which depth filtration is performed according to the following equation:

$$Flux ( L M H ) = \frac{Volume ( L )}{Time ( h ) * Surface area ( cm 2 )}$$

wherein the volume is the total preparation volume and the surface area is the surface area of the filter.

In another embodiment, the depth filtration is performed at a flux of between 100 and 600 LMH, between 200 and 400 LMH, between 250 and 350 LMH, or around 300 LMH.

During the step of depth filtration, the pressure may be measured using a pressure sensor connected to a pressure monitor. The pressure sensor may be triggered at a pressure of 15 psi and terminate the step of depth filtration. The step of depth filtration may therefore occur at a pressure of less than 15 psi.

In another embodiment, the step of depth filtration uses a filter which is an organic filter. Optionally, the filter comprises cellulose fibres and an inorganic filter aid. Optionally, the filter used in the depth filtration has a micron rating falling within the range of 0.1 to 10 μm. Optionally, the filter has a micron rating of 0.2 to 1.1 μm. Optionally, the filter is a C0HC filter.

In another embodiment, the step of depth filtration increases the viral genome titre of the preparation comprising recombinant AAV.

In another embodiment, the preparation comprising recombinant AAV has increased viral genome titre when compared to a preparation comprising recombinant AAV produced by a corresponding method or use not comprising a step of performing depth filtration. The preparation comprising recombinant AAV is the preparation that is produced by the methods and uses of the invention. Accordingly, to determine whether a method or use produces a preparation comprising recombinant AAV increased viral genome titre, the viral genome titre of the preparation should be measured once the method or use has been completed, i.e. after the step of performing depth filtration if the method or use comprises further subsequent steps once those further subsequent steps have been completed, and compared to the viral genome titre of a preparation made using an equivalent method lacking a step of performing depth filtration.

In another embodiment, the step of depth filtration increases the viral genome titre of the preparation comprising recombinant AAV by at least 5%, at least 10%, at least 15%, at least 20%, or at least 25%, when compared to a preparation comprising recombinant AAV produced by a corresponding method or use not comprising the step of depth filtration. To determine whether a step of performing depth filtration increases the viral genome titre of the preparation, the viral genome titre of the preparation should be measured immediately after the step is performed, i.e., before any steps subsequent to the step of performing depth filtration. The viral genome titre should be compared to the viral genome titre measured immediately after an equivalent point in an equivalent method or use lacking a step of performing depth filtration. For example, to determine if a step of performing depth filtration has increased the viral genome titre of a preparation comprising recombinant AAV, the viral genome titre should be measured immediately after the step of depth filtration and compared to the viral genome titre measured immediately after an equivalent point in an equivalent method or use lacking a step of performing depth filtration.

### Viral Genome Capsid Ratio

The viral genome/capsid ratio (as measured as a percentage of the total number of particles that are full particles) may be determined using qPCR to determine the number of vector genomes (as discussed in the section headed Viral genome titre assay), and using a capsid-specific ELISA to measure the total number of particles (as discussed in the section headed Capsid titre assay). Optionally, the viral genome/capsid ratio may be calculated by dividing the viral genome titre as measured by qPCR by the capsid titre as measured by ELISA and multiplying the result by 100%, wherein the units, in which the viral genome titre and the capsid titre are expressed, are the same.

Methods and uses of the present invention may increase capsid titre more than the viral genome titre, resulting in a reduced viral genome/capsid ratio. In one embodiment, the methods or uses of the present invention do not significantly reduce the viral genome/capsid ratio of the preparation comprising recombinant AAV when compared to a corresponding method or use comprising the step of mechanical lysis at a pressure of 5 kpsi. In one embodiment, the methods or uses of the present invention do not significantly reduce the viral genome/capsid ratio of the preparation comprising recombinant AAV when compared to a corresponding method or use comprising the step of mechanical lysis at a pressure of 10 kpsi. The preparation comprising recombinant AAV is the preparation that is produced by the methods and uses of the invention. Accordingly, to determine whether a method or use produces a preparation comprising recombinant AAV with a reduced viral genome/capsid ratio, the viral genome titre and capsid titre of the preparation should be measured once the method or use has been completed, i.e. after the step of performing mechanical lysis and if the method or use comprises further subsequent steps once those further subsequent steps have been completed, and compared to the viral genome/capsid ratio of a preparation made using an equivalent method comprising a step of performing mechanical lysis and/or lacking a step of performing mechanical lysis.

In another embodiment, the methods or uses of the present invention reduce the viral genome/capsid ratio of the preparation comprising recombinant AAV by no more than 10% when compared to a corresponding method or use comprising the step of mechanical lysis at a pressure of 5 kpsi.

In another embodiment, the methods or uses of the present invention reduce the viral genome/capsid ratio of the preparation comprising recombinant AAV by no more than 10% when compared to a corresponding method or use comprising the step of mechanical lysis at a pressure of 10 kpsi.

In another embodiment, the methods or uses of the present invention reduce the viral genome/capsid ratio of the preparation comprising recombinant AAV by no more than 15% when compared to a corresponding method or use comprising the step of mechanical lysis at a pressure of 5 kpsi.

In another embodiment, the methods or uses of the present invention reduce the viral genome/capsid ratio of the preparation comprising recombinant AAV by no more than 15% when compared to a corresponding method or use comprising the step of mechanical lysis at a pressure of 10 kpsi.

In one embodiment of the present invention, the step of depth filtration increases the viral genome/capsid ratio of the preparation comprising recombinant AAV.

In another embodiment, the preparation comprising recombinant AAV has an increased viral genome/capsid ratio when compared to a preparation comprising recombinant AAV produced by a corresponding method or use not comprising a step of performing depth filtration.

In another embodiment, the step of depth filtration increases the viral genome/capsid ratio of the preparation comprising recombinant AAV by at least 1%, at least 2%, at least 3%, or at least 4%. To determine whether a step of performing depth filtration increases the viral genome/capsid ratio of the preparation, the viral genome titre and capsid titre of the preparation should be measured immediately after the step of depth filtration is performed, i.e., before any steps subsequent to the step of performing depth filtration. The viral genome titre should be compared to the viral genome titre measured immediately after an equivalent point in an equivalent method or use lacking a step of performing depth filtration. For example, to determine if a step of performing depth filtration has increased the viral genome/capsid ratio of a preparation comprising recombinant AAV, the viral genome titre and capsid titre should be measured immediately after the step of depth filtration and the viral genome/capsid ratio calculated and compared to the viral genome/capsid ratio calculated from the viral genome titre and capsid titre measured at an equivalent point in an equivalent method or use lacking a step of performing depth filtration.

### Reduced Turbidity

Turbidity is a measure of the opacity of a solution. Impurities, such as cell debris or precipitated macromolecules, may increase turbidity by more than correctly folded proteins in solution e.g. AAV capsids. The turbidity of a preparation may therefore be used as an indirect measure of the impurities present in a preparation comprising recombinant AAV. It is often advantageous to reduce the turbidity of a preparation comprising recombinant AAV. The skilled person would understand that there are many methods to measure the turbidity of a preparation. For example, turbidity may be measured using the TL2360 (Hach) device. A suitable method for measuring the turbidity of a preparation comprising recombinant AAV is set out in the Examples.

In one embodiment, the preparation comprising recombinant AAV has reduced turbidity.

In another embodiment, the preparation comprising recombinant AAV has reduced turbidity compared to a preparation comprising recombinant AAV produced by a method not comprising the step of depth filtration.

In another embodiment, the preparation comprising recombinant AAV has a turbidity of less than 50 Nephelometric Turbidity Units (NTU), less than 40 NTU, less than 30 NTU, or less than 20 NTU.

### Sterile Filtration

The uses and methods of the invention may further comprise a step of sterile filtration. Sterile filtration comprises the step of passing a preparation through a sterile filter, optionally a bottle-top filter. A filter used in sterile filter has a micron rating, meaning that only particles which are smaller than said rating may pass through the filter, while larger particles will be retained. Optionally, the sterile filtration is performed using a 0.22 μm filter. Optionally, the step of sterile filtration reduces the turbidity of the preparation comprising recombinant AAV to 5 NTU or lower. Optionally, the step of sterile filtration reduces the turbidity of the preparation comprising recombinant AAV to 3 NTU or lower. Optionally, the step of sterile filtration reduces the turbidity of the preparation comprising recombinant AAV to 1 NTU or lower.

### Purifying the Recombinant AAV

The method or use of the invention may further comprise a step of purifying the rAAV. In general, a step of purifying the rAAV will involve increasing the concentration of the rAAV compared to other components of the preparation. Optionally, the step of purifying the rAAV results in a concentrated rAAV preparation. Optionally, the step of purifying the rAAV results in an isolated rAAV.

Any suitable purification method may be used. Optionally, the step of purifying the rAAV is carried out using known technique such as gradient density centrifugation (such as CsCl or Iodixanol gradient density centrifugation), filtration, ion exchange chromatography, size exclusion chromatography, affinity chromatography and hydrophobic interaction chromatography or a combination of some or all of these techniques.

Optionally, the method comprises further concentrating the rAAV using ultracentrifugation, tangential flow filtration, and/or gel filtration.

In one embodiment, the method or use further comprises a step of purifying the rAAV. Optionally, the step of purification occurs after the step of mechanical lysis. Optionally, the step of purification occurs after the step of depth filtration. Optionally, the step of purification occurs after the step of mechanical lysis and after the step of depth filtration. Optionally, the step of purification occurs after the step of sterile filtration. Optionally, the step of purification occurs after the step of mechanical lysis, after the step of depth filtration, and after the step of sterile filtration.

### Pharmaceutical Compositions

The methods and uses of the invention may further comprise the step of formulating the preparation comprising recombinant AAV with a pharmaceutically acceptable excipient. The pharmaceutically acceptable excipients may comprise carriers, diluents and/or other medicinal agents, pharmaceutical agents or adjuvants, etc. Optionally, the pharmaceutically acceptable excipients comprise saline solution. Optionally, the pharmaceutically acceptable excipients comprise human serum albumin. Optionally, the preparation is suitable for human administration.

In one aspect of the present invention, the method or use further comprises a step of formulating the preparation comprising recombinant AAV with a pharmaceutically acceptable excipient.

In one aspect, the present invention provides a pharmaceutical composition or preparation comprising recombinant AAV obtainable by the methods or uses of the present invention.

In one aspect, the present invention provides a pharmaceutical composition or preparation comprising recombinant AAV obtained by the methods or uses of the present invention.

### Methods of Treatment

Further provided is a method for the treatment or prevention of a disease, comprising administering the preparation or pharmaceutical composition of the invention to a patient in need thereof. Further provided is the preparation or the pharmaceutical composition of the invention for use in a method of treating or preventing a disease. Further provided is the use of the preparation or the pharmaceutical composition of the invention in the manufacture of a medicament for the treatment or prevention of a disease.

Optionally, the method of treating or preventing a disease comprises administering an effective amount of the preparation or pharmaceutical composition of the invention to a patient. Optionally, the patient suffers from a genetic disorder. Optionally, the disease is a genetic disorder. For the purposes of the present invention, a genetic disorder is any disorder associated with a mutation in a gene. Optionally, the genetic disorder is a genetic disorder that can be treated by gene therapy, for example using AAV as a gene therapy vector. Optionally, the genetic disorder is selected from the group consisting of haemophilia A, haemophilia B, Gaucher's disease and Fabry disease.

A number of embodiments of the invention have been described. Nevertheless, one skilled in the art, without departing from the spirit and scope of the invention, can make various changes and modifications of the invention to adapt it to various usages and conditions. Accordingly, the following examples are intended to illustrate but not limit the scope of the invention claimed in any way.

### EXAMPLES

### Example 1

### Cell Cultivation and Transfection

HEK293 cells were maintained in suspension culture under standard conditions at 37° C., and 5% v/v CO2 in BalanCD medium (Irvine Scientific, catalogue number 91165) supplemented with 4 mM L-glutamine. Cellular viability was maintained at >80%.

0.6-0.7×10\hat{ }6 viable HEK293 cells/mL were seeded in the bioreactor. For transfection, vector plasmid comprising an engineered cap gene and an expression cassette of interest flanked by inverted terminal repeats (ITRs) and a helper plasmid comprising rep genes and adenoviral helper genes were mixed with PEIpro transfection reagent according to manufacturer's instructions. 1-1.2×10\hat{ }6 viable HEK293 cells/mL were transfected and cultured for 72 hrs.

### Cell Lysis

Following cell culture, the cells were lysed by microfluidisation in the suspension culture medium using the Constant System LTD—CF1 (cylinder diameter 18 mm) according to manufacturer's instructions. Lysis was carried out at 5° C. in continuous flow. At each shot, the sample is momentarily trapped in the high-pressure cylinder and acting pressures instantly move from ambient to the set pressure. Different pressures were tested as indicated, ranging from 2 kilo pound per square inch (kpsi) to 40 kpsi. Cells were lysed in a single pass.

### Denarase Treatment

Following microfluidization, digestion of non-packaged DNA was performed on the lysate with the recombinant endonuclease Denarase® (c-LEcta, Cat No. 20804) at 20 U/ml. The samples were treated overnight with recombinant endonuclease at room temperature while mixing.

### Depth Filtration

Following microfluidisation and digestion of the non-packaged DNA, the sample was depth filtered. Different depth filters from Millipore were used, including the synthetic C0SP filter, which is made of polyacrylic fibres and a silica, as well as the organic C0HC filter, which is made out of cellulose fibres and an inorganic filter aid (see Table 1). These filters are scalable and their use removes cell debris as well as aggregated proteins, decreasing the turbidity of the samples.

Table 1: Overview of the different filters used for depth filtration.

| | | | Micron | Manufacturer |
|---|---|---|---|---|
| | | Size | rating | & catalogue |
| Filter | Material | [cm²] | [μm] | number |
| C0SP | synthetic - polyacrylic | 23 | 0.2-1.1 | Merck Millipore |
| | fibers | | | M C0SP |
| | and silica | | | 23C L 3 |
| C0HC | organic - cellulose | 23 | 0.2-1.1 | Merck Millipore |
| | fibers and | | | M C0HC |
| | inorganic filter aid | | | 23C L 3 |

The filtration was performed at a Flux of 300 LMH. The Flux can be determined with the following calculation:

$$Flux ( L M H ) = \frac{Volume ( L )}{Time ( h ) * Surface area ( cm 2 )}$$

Thereby, the pressure is measured by a single-use pressure sensor connected to a pressure monitor. The threshold for the maximum pressure was 15 psi. After depth filtration, the turbidity was measured using the TL2360 (Hach) device to check if any breakthrough in particles occurred or not. Values are indicated as Nephelometric Turbidity Unit (NTU).

Quantification of rAAV Vector Genomes (Viral Genome Titre)

The AAV vector genome assay is based on a quantitative polymerase chain reaction (qPCR) specific for a sequence of the rAAV expression cassette.

Cell lysate test samples were subjected to a nuclease treatment procedure in order to remove non-packed vector genomes prior to performing the qPCR. To that aim the samples were pre-diluted 1:250 in nuclease-free water containing 0.124% Pluronic F-68. 25 μl of the pre-dilution were used for the digest with 2 units of Turbo DNase (ThermoFisher Scientific, Waltham, USA) and 1× Turbo DNase reaction buffer, resulting in a total reaction volume of 29 μl. Incubation was performed for 1 h at 37° C. Afterwards, 1 volume of 0.4 M NaOH was added and the samples were incubated for 45 min at 65° C. 1035 μl nuclease-free water supplemented with 0.1% Pluronic F-68 were added together with 30 μl 0.4 M HCl. To control for the quality of the Turbo DNase digest, a trending control containing unpurified cell lysate with a known AAV vector genome titre and spike-in controls using plasmid DNA carrying the sequence of the expression cassette were measured in parallel.

Per sample, 12.5 μl QuantiFast SYBR Green PCR Master Mix (Qiagen, Venlo, Netherlands) were mixed with 0.75 μl of qPCR primer working stock solution (containing 10 μM of each primer) and filled up to a volume of 20 μl with nuclease-free water. 5 μl of Turbo DNase treated cell lysate or purified virus test sample were added to the mix (total reaction volume 25 μl, final primer concentration in the reaction 300 nM each) and qPCR was performed in a CFX 96 Touch Real Time PCR cycler (Bio-Rad Laboratories Inc., Hercules, USA) with following program steps: 95° C. 5 min; 39 cycles (95° C. 10 sec, 60° C. 30 sec, plate read); 95° C. 10 sec; 60-95° C. (+0.5° C./step), 10 sec; plate read. To control for the quality of the qPCR, a trending control with known AAV vector genome titre was measured in parallel. To check for contamination, a no template control (NTC, 5 μl H2O) was also included. Standard row, test samples and controls were measured in triplicates for each dilution. Purified virus test samples and trending control were generally measured in 3 different dilutions in EB buffer (10 mM Tris-Cl, pH 8.5). Turbo DNase treated cell lysate test samples were directly used in the qPCR without any further dilution. Data were analysed using the CFX Manager™ Software 3.1 (Bio-Rad Laboratories Inc.).

Melting curve analysis confirmed the presence of only one amplicon. Amplification results in nascent double stranded DNA amplicons detected with the fluorescent intercalator SYBR Green to monitor the PCR reaction in real time. Known quantities of the expression cassette genetic material, in the form of a linearised plasmid, were serially diluted to create a standard curve and sample vector genome titre was interpolated from the standard curve.

Quantification of rAAV Particles (Capsid Titre)

The AAV2 Titration ELISA method is a measure of total AAV particles (capsids) and is based on a commercially available kit (Progen™, Heidelberg, Germany; catalogue number PRATV). This sandwich immunometric technique utilises monoclonal antibody A20 (Wobus et al (2000), J Virol, 74:9281-9293) for both capture and detection. The antibody is specific for a conformational epitope present on assembled capsids of serotypes AAV2, AAV3, and the engineered capsid used in these experiments.

The AAV2 Titration ELISA kit was used to quantify total AAV particles in cell lysates and purified virus preparations according to the manufacturer's instructions. In brief, 100 μl diluted AAV2 Kit Control, test samples, or trending control of engineered capsid with known total particle titre were added per well of a microtitre plate coated with monoclonal antibody A20 and incubated for 1 hour at 37° C. Standard row, test samples and controls were measured in duplicates for each dilution. In a second step, 100 μl of pre-diluted biotin-conjugated monoclonal antibody A20 (1:20 in Assay Buffer [ASSB]) were added and incubated for 1 h at 37° C. Then, 100 μl of a pre-diluted streptavidin peroxidase conjugate (1:20 in ASSB) were added and incubated for 1 h at 37° C. 100 μl substrate solution (TMB [Tetramethylbenzidine]) were added and after incubation for 15 min, the reaction was stopped using 100 μl stop solution. The absorbance was measured photochemically at 450 nm using the SpectraMax M3 microplate reader (Molecular Devices, San Jose, USA). Data were analysed with the SoftMax Pro 7.0 Software (Molecular Devices).

The test samples were diluted into the assay range and AAV total particle concentrations were determined by interpolation using the standard curve which was prepared using the provided AAV2 Kit Control. ASSB was used as blank.

### Vector Genome to Total Particle Ratio

The ratio of vector genomes to total AAV particles is expressed as a percentage. This is based on the vector genome titre (determined by qPCR, as described above) and the number of total AAV particles (determined by the capsid ELISA, as described above).

### Results

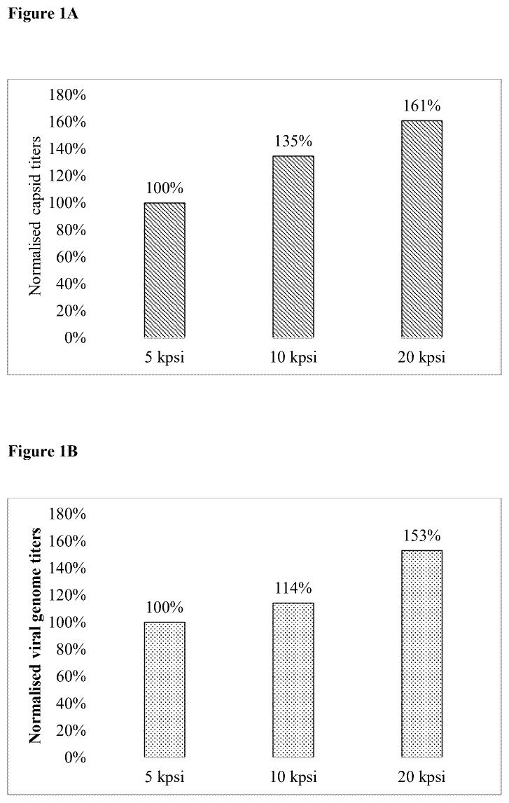

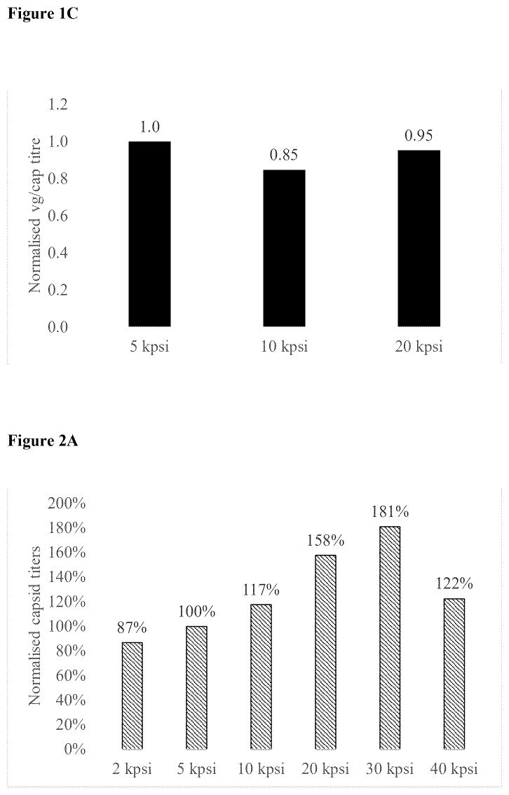

Using microfluidisation, HEK293 suspension cells can be lysed efficiently. It was shown that increasing the pressure used during microfluidisation (MF) for lysis of the cells resulted in increased viral genome (vg) and capsid (cap) titres. Compared to lysis at 5 kpsi, capsid titres were increased with pressure at 10 kpsi and 20 kpsi (see FIG. 1A). Similarly, viral genome titres were increased at pressures of 10 kpsi and 20 kpsi, respectively (see FIG. 1i), while the vg/cap ratio was almost unchanged (see FIG. 1C), demonstrating that by using higher pressure in the mechanical lysis step overall more capsids can be extracted from the cells without compromising the vg to capsid ratio.

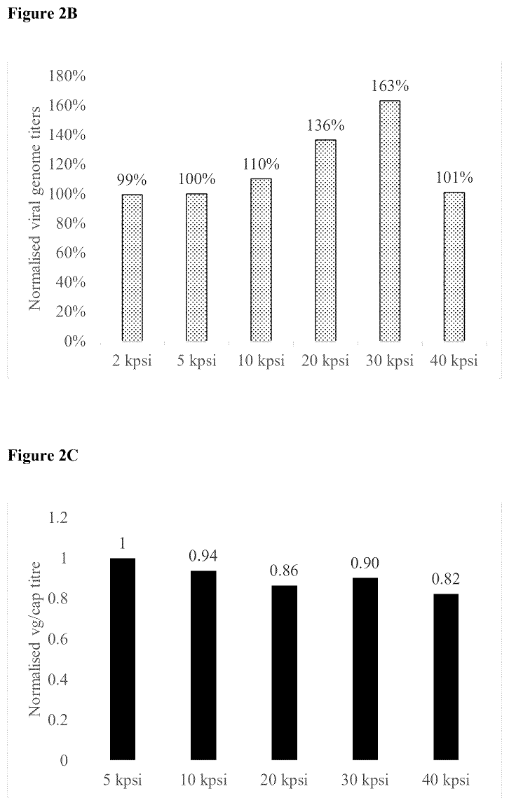

Further increases were achieved at a pressure of 30 kpsi for capsid titres (see FIG. 2A) and viral genome titres (see FIG. 2B). A drop in recovery of the rAAV particles was observed at 40 kpsi. The vg/cap ratio was almost unchanged between the different pressures (see FIG. 2C), indicating that overall better yields can be obtained at pressures of 10 kpsi or higher, in particular at pressures of 20 kpsi or higher.

Taken together, increased capsid and vg titre yields were achieved using mechanical lysis in the form of microfluidisation at pressures of 10 kpsi or higher without a significant decrease in the vg/cap ratio. This demonstrates that the cells are lysed efficiently using microfluidisation at pressures of 10 kpsi or higher compared to the more commonly used pressures of around 5 kpsi. Therefore, the yield of rAAV particles can be increased using microfluidisation at higher pressures without damage to the capsids. Cells were lysed in a single pass.

### Example 2

### C0SP and C0HC Filter

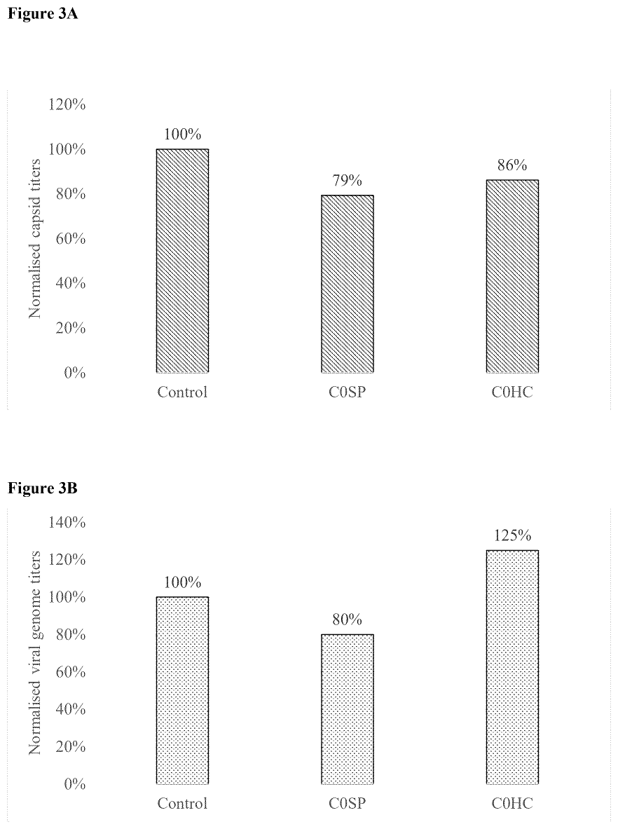

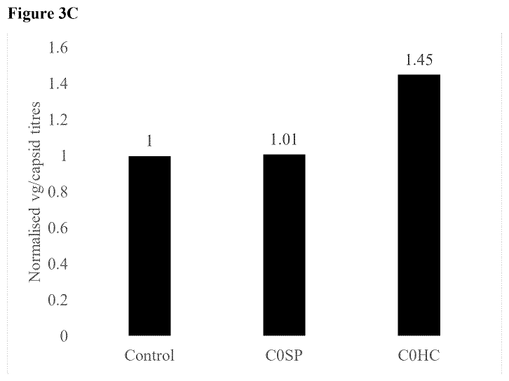

To decrease the turbidity, depth filtration was tested using the following filters: the synthetic C0SP and the organic C0HC filter, both with an area of 23 cm² and a micron rating of 0.2-1.1 μm. For both filters each a volume of 0.9 L of lysate was processed. In comparison to the control (cells lysed with MF at 20 kpsi without depth filtration), the C0SP and C0HC depth filtration resulted in similar cap titres (see FIG. 3A), indicating that the viral particles were not adsorbed by the filters. Furthermore, the vg titres (see FIG. 3B) and vg/cap ratios (see FIG. 3C) were decreased for the synthetic C0SP filter and increased for the organic C0HC filter. Taken together, overall best results were achieved for MF at 20 kpsi and depth filtration using an organic C0HC filter (see FIG. 3). Turbidity was reduced to 8.21 NTU and 17.7 NTU for the C0SP and the C0HC filters, respectively. For all depth filters used, the pressure was not increased above the threshold of 15 psi. The HC type filter was better in terms of performance and recovery in comparison to the SP type.

### Example 3

### Microfluidisation, Depth Filtration and Sterile Filtration at 50 L Scale

The new lysis and clarification method using mechanical lysis followed by depth filtration was successfully scaled up to the 50 L scale (see Table 2). At the 50 L scale, a step of sterile filtration using a 0.22 μm bottle-top filter was added. Recoveries ranging from 80-90% were achieved, as well as final turbidity values ranging from 1-5 NTU, demonstrating the scalability of the method.

Table 2: Results for three 50 L runs. Capsid and vg titre recoveries from clarification are shown for rAAV comprising a transgene of 2.3 kb (run #1 and #2) and 3.2 kb (run #3), including final turbidity values. Recovery values are shown relative to recovery from clarification.

| | Capsid titre | Viral genome titre | |
|---|---|---|---|
| | recovery | recovery | Final turbidity |
| 50 L run #1 | 87% | 90% | 1 NTU |
| 50 L run #2 | 89% | 84% | 5 NTU |
| 50 L run #3 | 93% | 86% | 3 NTU |

The invention described herein also relates to the following aspects:
1. A method for producing a preparation comprising recombinant adeno-associated virus (AAV), wherein the method comprises a step of performing mechanical lysis on mammalian producer cells comprising the recombinant AAV at a pressure of 7 kilo pounds per square inch (kpsi) or greater.
2. A method for increasing the viral genome titre and/or capsid titre of a preparation comprising recombinant adeno-associated virus (AAV), wherein the method comprises a step of performing mechanical lysis on mammalian producer cells comprising the recombinant AAV at a pressure of 7 kilo pounds per square inch (kpsi) or greater.
3. Use of mechanical lysis for producing a preparation comprising recombinant adeno-associated virus (AAV), wherein the mechanical lysis is performed on mammalian producer cells comprising the recombinant AAV at a pressure of 7 kilo pounds per square inch (kpsi) or greater.
4. Use of mechanical lysis for increasing the viral genome titre and/or capsid titre of a preparation comprising recombinant adeno-associated virus (AAV), wherein the mechanical lysis is performed on mammalian producer cells comprising the recombinant AAV at a pressure of 7 kilo pounds per square inch (kpsi) or greater.
5. The method or use of any one of aspects 1-4, wherein the step of mechanical lysis is carried out by microfluidisation.
6. The method or use of aspect 5, wherein the step of microfluidisation consists of three or fewer or two or fewer passes.
7. The method or use of aspect 5 or 6, wherein the step of mechanical lysis consists of one pass.
8. The method or use of any one of aspects 1-7, wherein the mammalian producer cells are HEK293 cells, HEK293T cells, HEK293SF cells, HEK293-F cells, HEK293-derived cells, CHO cells, HeLa cells, HeLa S3 cells, HEK293EBNA cells, CAP cells, CAP-T cells, AGE1.CR cells, PerC6 cells, C139 cells, EB66 cells, BHK cells, COS cells, Vero cells or A549 cells.
9. The method or use of any one of aspects 1-8, wherein the mammalian producer cells are selected from the group comprising HEK293 cells, HEK293T cells, HEK293SF cells, HEK293-F cells, HEK293-derived cells, CHO cells, HeLa cells, HeLa S3 cells, HEK293EBNA cells, CAP cells, CAP-T cells, AGE1.CR cells, PerC6 cells, C139 cells, EB66 cells, BHK cells, COS cells, Vero cells and A549 cells.
10. The method or use of any one of aspects 1-9, wherein the mammalian producer cells are human cells.
11. The method or use of any one of aspects 1-10, wherein the mammalian producer cells are HEK293, HEK293SF cells, HEK293-F cells, HEK293-derived cells or HEK293T cells.
12. The method or use of any one of aspects 1-11, further comprising a step of culturing the mammalian producer cells in cell culture medium before the step of performing mechanical lysis.
13. The method or use of aspect 12, wherein the step of mechanical lysis occurs on mammalian producer cells comprising recombinant AAV in the cell culture medium.
14. The method or use of any one of aspects 1-13, further comprising a step of harvesting the mammalian producer cells.
15. The method or use of aspect 14, wherein the step of harvesting the mammalian producer cells occurs before the step of mechanical lysis.
16. The method or use of any one of aspects 1-15, wherein the pressure is 10 kpsi or greater, 11 kpsi or greater, 12 kpsi or greater, 13 kpsi or greater, 14 kpsi or greater, 15 kpsi or greater, 16 kspi or greater, 17 kpsi or greater, 18 kpsi or greater, 19 kpsi or greater, 20 kpsi or greater, 21 kpsi or greater, 22 kpsi or greater, 23 kpsi or greater, 24 kpsi or greater, 25 kpsi or greater, 26 kpsi or greater, 27 kpsi or greater, 28 kpsi or greater, 29 kpsi or greater, 30 kpsi or greater, 31 kpsi or greater, 32 kpsi or greater, 33 kpsi or greater, 34 kpsi or greater, or 35 kpsi or greater.
17. The method or use of any one of aspects 1-16, wherein the pressure is 15 kpsi or greater.
18. The method or use of any one of aspects 1-17, wherein the pressure is 20 kpsi or greater.
19. The method or use of any one of aspects 1-18, wherein the pressure is 25 kpsi or greater.
20. The method or use of any one of aspects 1-19, wherein the pressure is 30 kpsi or greater.
21. The method or use of any one of aspects 1-20, wherein the pressure is 35 kpsi or greater.
22. The method of any one of aspects 1-21, wherein the pressure is less than 42.5 kpsi.
23. The method of any one of aspects 1-22, wherein the pressure is less than 40 kpsi.
24. The method or use of any one of aspects 1-23, wherein the pressure is less than 37.5 kpsi.
25. The method or use of any one of aspects 1-24, wherein the pressure is between 10 kpsi and 40 kpsi, between 15 kpsi and 40 kpsi, between 20 kpsi and 40 kpsi, between 25 kpsi and 40 kpsi, between 30 kpsi and 40 kpsi, between 35 kpsi and 40 kpsi, between 10 kpsi and 35 kpsi, between 15 kpsi and 35 kpsi, between 20 kpsi and 35 kpsi, between 25 kpsi and 35 kpsi, between 30 kpsi and 35 kpsi, between 10 kpsi and 30 kpsi, between 15 kpsi and 30 kpsi, between 15 kpsi and 25 kpsi, between 20 kpsi and 30 kpsi, or between 25 kpsi and 30 kpsi, or wherein the pressure is around 10 kpsi, around 15 kpsi, around 20 kpsi, around 25 kpsi, or around 30 kpsi.
26. The method or use of any one of aspects 1-25, wherein the pressure is between 10 kpsi and 40 kpsi.
27. The method or use of any one of aspects 1-26, wherein the pressure is between 20 kpsi and 40 kpsi.
28. The method or use of any one of aspects 1-27, wherein the pressure is between 20 kpsi and 30 kpsi.
29. The method or use of any one of aspects 1-28, wherein the mammalian producer cells have been transfected with one or more plasmids comprising an AAV cap gene and/or AAV rep genes, and optionally helper genes, e.g. adenoviral helper genes or helper genes derived from herpes simplex virus (HSV), and at least one inverted terminal repeat (ITR).
30. The method or use of any one of aspects 1-28, further comprising a step of transfecting the mammalian producer cells with one or more plasmids comprising an AAV cap gene and/or AAV rep genes, and optionally helper genes, e.g. adenoviral helper genes or helper genes derived from herpes simplex virus (HSV), and at least one inverted terminal repeat (ITR) before the step of performing mechanical lysis.
31. The method or use of aspect 29 or 30, wherein the one or more plasmids further comprise an expression cassette comprising a transgene between two ITRs.
32. The method or use of any one of aspects 1-31, wherein the mammalian producer cells comprise sufficient genetic material for the recombinant AAV to propagate.
33. The method or use of any one of aspects 1-32, wherein the mammalian producer cells comprise:
(i) a rep 52 gene;
(ii) a rep 40 gene;
(iii) a rep 68 gene;
(iv) a cap gene;
(v) a viral associated (VA) nucleic acid;
(vi) an E2a gene;
(vii) an E4 gene;
(viii) an E1A gene; and
(ix) a polynucleotide comprising an expression cassette comprising a transgene between two ITRs.
34. The method or use of any one of aspects 5-33, wherein the microfluidisation occurs at a temperature of 15° C. or lower, 10° C. or lower, 8° C. or lower, 5° C. or lower, between 5° C. and 10° C., or between 0° C. and 5° C.
35. The method or use of any one of aspects 5-34, wherein the microfluidisation occurs at 5° C. or lower.
36. The method or use of any one of aspects 1-35, wherein:
(a) the preparation comprising recombinant AAV has increased viral genome titre; and/or
(b) the method or use is a method or use for increasing the viral genome titre of the preparation comprising recombinant AAV; and/or
(c) the step of performing mechanical lysis increases the viral genome titre of the preparation comprising recombinant AAV.
37. The method or use of any one of aspects 1-36, wherein the preparation comprising recombinant AAV has increased viral genome titre when compared to a preparation comprising recombinant AAV produced by a corresponding method or use comprising a step of performing mechanical lysis at a pressure of 5 kpsi.
38. The method or use of any one of aspects 1-37, wherein:
(a) the preparation comprising recombinant AAV has at least 10%, at least 20%, at least 30%, at least 40%, at least 50%, or at least 60% increased viral genome titre;
(b) the method or use is a method or use for increasing the viral genome titre of the preparation comprising recombinant AAV by at least 10%, at least 20%, at least 30%, at least 40%, at least 50%, or at least 60%; or
(c) the step of performing mechanical lysis increases the viral genome titre of the preparation comprising recombinant AAV by least 10%, at least 20%, at least 30%, at least 40%, at least 50%, or at least 60%,
when compared to a preparation comprising recombinant AAV produced by a corresponding method or use comprising a step of performing mechanical lysis at a pressure of 5 kpsi.
39. The method or use of any one of aspects 1-38, wherein:
(a) the preparation comprising recombinant AAV has at least 10% increased viral genome titre;
(b) the method or use is a method or use for increasing the viral genome titre of the preparation comprising recombinant AAV by at least 10%; or
(c) the step of performing mechanical lysis increases the viral genome titre of the preparation comprising recombinant AAV by least 10%,
when compared to a preparation comprising recombinant AAV produced by a corresponding method or use comprising a step of performing mechanical lysis at a pressure of 5 kpsi.
40. The method or use of any one of aspects 1-39, wherein:
(a) the preparation comprising recombinant AAV has at least 30% increased viral genome titre;
(b) the method or use is a method or use for increasing the viral genome titre of the preparation comprising recombinant AAV by at least 30%; or
(c) the step of performing mechanical lysis increases the viral genome titre of the preparation comprising recombinant AAV by least 30%,
when compared to a preparation comprising recombinant AAV produced by a corresponding method or use comprising a step of performing mechanical lysis at a pressure of 5 kpsi.
41. The method or use of any one of aspects 1-40, wherein the viral genome titre is measured by qPCR.
42. The method or use of aspect 41, wherein the viral genome titre is measured by ddPCR.
43. The method or use of any one of aspects 1-42, wherein:
(a) the preparation comprising recombinant AAV has increased capsid titre; and/or
(b) the method or use is a method or use for increasing the capsid titre of the preparation comprising recombinant AAV; and/or
(c) the step of performing mechanical lysis increases the capsid titre of the preparation comprising recombinant AAV.
44. The method or use of any one of aspects 1-43, wherein the preparation comprising recombinant AAV has increased capsid titre when compared to a preparation comprising recombinant AAV produced by a corresponding method or use comprising a step of performing mechanical lysis at a pressure of 5 kpsi.
45. The method or use of any one of aspects 1-44, wherein:
(a) the preparation comprising recombinant AAV has at least 10%, at least 20%, at least 30%, at least 40%, at least 50%, at least 60%, at least 70%, or at least 80% increased capsid titre;
(b) the method or use is a method or use for increasing the capsid titre of the preparation comprising recombinant AAV by at least 10%, at least 20%, at least 30%, at least 40%, at least 50%, at least 60%, at least 70%, or at least 80%; or
(c) the step of performing mechanical lysis increases the capsid titre of the preparation comprising recombinant AAV by at least 10%, at least 20%, at least 30%, at least 40%, at least 50%, at least 60%, at least 70%, or at least 80%, when compared to a preparation comprising recombinant AAV produced by a corresponding method or use comprising a step of performing mechanical lysis at a pressure of 5 kpsi.
46. The method or use of any one of aspects 1-45, wherein:
(a) the preparation comprising recombinant AAV has at least 10% increased capsid titre;
(b) the method or use is a method or use for increasing the capsid titre of the preparation comprising recombinant AAV by at least 10%; or
(c) the step of performing mechanical lysis increases the capsid titre of the preparation comprising recombinant AAV by at least 10%,
when compared to a preparation comprising recombinant AAV produced by a corresponding method or use comprising a step of performing mechanical lysis at a pressure of 5 kpsi.
47. The method or use of any one of aspects 1-46, wherein:
(a) the preparation comprising recombinant AAV has at least 50% increased capsid titre;
(b) the method or use is a method or use for increasing the capsid titre of the preparation comprising recombinant AAV by at least 50%; or
(c) the step of performing mechanical lysis increases the capsid titre of the preparation comprising recombinant AAV by at least 50%,
when compared to a preparation comprising recombinant AAV produced by a corresponding method or use comprising a step of performing mechanical lysis at a pressure of 5 kpsi.
48. The method or use of any one of aspects 1-47, wherein the capsid titre is measured by ELISA.
49. The method or use of any one of aspects 1-48, wherein the method or use further comprises a step of endonuclease treatment.
50. The method or use of aspect 49, wherein the step of endonuclease treatment occurs after the step of mechanical lysis.
51. The method or use of any one of aspects 1-50, wherein the method or use further comprises a step of depth filtration.
52. The method or use of aspect 51, wherein the step of depth filtration occurs after the step of mechanical lysis.
53. The method or use of aspect 51 or aspect 52, wherein the step of depth filtration occurs after the step of endonuclease treatment.
54. The method or use of any one of aspects 51-53, wherein the depth filtration is performed at a flux of between 100 and 600 LMH, between 200 and 400 LMH, between 250 and 350 LMH, or around 300 LMH.
55. The method or use of any one of aspects 51-54, wherein the step of depth filtration uses a filter which is an organic filter.
56. The method or use of aspect 55, wherein the filter comprises cellulose fibres and an inorganic filter aid.
57. The method or use of any one of aspects 51-56, wherein the step of depth filtration uses a filter that has a micron rating falling within the range of 0.1 to 10 μm.
58. The method or use of aspect 57, wherein the filter has a micron rating of 0.2 to 1.1 μm.
59. The method or use of aspect 57 or aspect 58, wherein the filter is a C0HC filter.
60. The method or use of any one of aspects 51-59, wherein the step of depth filtration increases the viral genome titre of the preparation comprising recombinant AAV.
61. The method or use of any one of aspects 51-60, wherein the preparation comprising recombinant AAV has increased viral genome titre when compared to a preparation comprising recombinant AAV produced by a corresponding method or use not comprising a step of performing depth filtration.
62. The method or use of any one of aspects 51-61, wherein the step of depth filtration increases the viral genome titre of the preparation comprising recombinant AAV by at least 5%, at least 10%, at least 15%, at least 20%, or at least 25%, when compared to a preparation comprising recombinant AAV produced by a corresponding method or use not comprising the step of depth filtration.
63. The method or use of any one of aspects 51-62, wherein the step of depth filtration increases the viral genome titre of the preparation comprising recombinant AAV by at least 10%, when compared to a preparation comprising recombinant AAV produced by a corresponding method or use not comprising the step of depth filtration.
64. The method or use of any one of aspects 51-63, wherein the step of depth filtration increases the viral genome titre of the preparation comprising recombinant AAV by at least 25%, when compared to a preparation comprising recombinant AAV produced by a corresponding method or use not comprising a step of performing depth filtration.
65. The method or use of any one of aspects 51-64, wherein the viral genome titre is measured by qPCR.
66. The method or use of aspect 65, wherein the viral genome titre is measured by ddPCR.
67. The method or use of any one of aspects 51-66, wherein the step of depth filtration increases the viral genome/capsid ratio of the preparation comprising recombinant AAV.
68. The method or use of any one of aspects 51-67, wherein the preparation comprising recombinant AAV has an increased viral genome/capsid ratio when compared to a preparation comprising recombinant AAV produced by a corresponding method or use not comprising a step of performing depth filtration.
69. The method or use of any one of aspects 51-68, wherein the step of depth filtration increases the viral genome/capsid ratio of the preparation comprising recombinant AAV by at least 1%, at least 2%, at least 3%, or at least 4%.
70. The method or use of any one of aspects 51-69, wherein the step of depth filtration increases the viral genome/capsid ratio of the preparation comprising recombinant AAV by at least 4%.
71. The method or use of any one of aspects 67-70, wherein the viral genome/capsid ratio is calculated by dividing the viral genome titre as measured by qPCR by the capsid titre as measured by ELISA and multiplying the result by 100%, wherein the units in which the viral genome titre and the capsid titre are expressed are the same.
72. The method or use of any one of aspects 51-71, wherein the preparation comprising recombinant AAV has reduced turbidity.
73. The method or use of any one of aspects 51-72, wherein the preparation comprising recombinant AAV has reduced turbidity compared to a preparation comprising recombinant AAV produced by a method not comprising the step of depth filtration.
74. The method or use of any one of aspects 51-73, wherein the preparation comprising recombinant AAV has a turbidity of less than 50 Nephelometric Turbidity Units (NTU), less than 40 NTU, less than 30 NTU, or less than 20 NTU.
75. The method or use of any one of aspects 51-74, wherein the preparation comprising recombinant AAV has a turbidity of less than 20 NTU.
76. The method or use of any one of aspects 51-75, further comprising a step of sterile filtration.
77. The method or use of aspect 76, wherein the sterile filtration is performed using a 0.22 μm filter.
78. The method or use of aspect 76 or aspect 77, wherein the preparation comprising recombinant AAV has a turbidity of 5 NTU or lower.
79. The method or use of any one of aspects 76-78, wherein the preparation comprising recombinant AAV has a turbidity of 3 NTU or lower.
80. The method or use of any one of aspects 76-79, wherein the preparation comprising recombinant AAV has a turbidity of 1 NTU or lower.
81. The method or use of any one of aspects 1-80, further comprising a step of purifying the recombinant AAV.
82. The method or use of any one of aspects 1-81, further comprising a step of formulating the preparation comprising recombinant AAV with a pharmaceutically acceptable excipient.
83. A preparation comprising recombinant AAV obtainable by the method or use of any one of aspects 1-82.
84. A preparation comprising recombinant AAV obtained by the method or use of any one of aspects 1-82.
85. A method for the treatment or prevention of a disease, comprising administering the preparation of aspect 83 or aspect 84 to a patient in need thereof.
86. A preparation according to aspect 83 or aspect 84, for use in a method of treating a disease.
87. The method of aspect 85 or the preparation of aspect 86, wherein the disease is a genetic disorder.

1-18. (canceled)

19. A method for producing a preparation comprising recombinant adeno-associated virus (AAV), wherein the method comprises a step of performing mechanical lysis on mammalian producer cells comprising the recombinant AAV at a pressure between 10 and 30, preferably between 15 and 30 0 kilo pounds per square inch (kpsi).

20. The method of claim 19, wherein the step of performing mechanical lysis is carried out by microfluidisation.

21. The method or use of claim 20, wherein the step of performing microfluidisation consists of one pass.

22. The method of claim 19, wherein the mammalian producer cells are HEK293 cells, HEK293T cells, HEK293SF cells, HEK293-F cells, HEK293-derived cells, CHO cells, HeLa cells, HeLa S3 cells, HEK293EBNA cells, CAP cells, CAP-T cells, AGE1.CR cells, PerC6 cells, C139 cells, EB66 cells, BHK cells, COS cells, Vero cells, or A549 cells.

23. The method of claim 19, further comprising a step of culturing the mammalian producer cells in cell culture medium before the step of performing mechanical lysis, and/or wherein the step of mechanical lysis occurs on the mammalian producer cells comprising recombinant AAV in the cell culture medium.

24. The method of claim 20, wherein the microfluidisation occurs at a temperature of 15° C. or lower, 10° C. or lower, 8° C. or lower, 5° C. or lower, between 5° C. and 10° C., or between 0° C. and 5° C.

25. The method of claim 19, wherein the preparation comprising recombinant AAV has at least 10%, at least 20%, at least 30%, at least 40%, at least 50%, or at least 60% increased viral genome titre and/or increased capsid titre when compared to a preparation comprising recombinant AAV produced by a corresponding method or use comprising a step of performing mechanical lysis at a pressure of 5 kpsi.

26. The method of claim 19, further comprises performing a step of endonuclease treatment, optionally wherein the step of endonuclease treatment occurs after the step of mechanical lysis.

27. The method of claim 26, further comprises performing a step of depth filtration.

28. The method of claim 27, wherein:
(i) the step of depth filtration occurs after the step of mechanical lysis; and/or
(ii) the step of depth filtration occurs after the step of endonuclease treatment; and/or
(iii) the depth filtration is performed at a flux of between 100 and 600 LMH, between 200 and 400 LMH, between 250 and 350 LMH, or around 300 LMH; and/or
(iv) the step of depth filtration uses a filter which is an organic filter, optionally wherein the filter comprises cellulose fibres and an inorganic filter aid; and/or
(v) the step of depth filtration uses a filter that has a micron rating falling within the range of 0.1 to 10 μm, optionally wherein the filter has a micron rating of 0.2 to 1.1 μm and/or wherein the filter is a C0HC filter.

29. The method of claim 28, wherein
(i) the step of depth filtration increases a viral genome titre of the preparation comprising recombinant AAV; and/or
(ii) the preparation comprising recombinant AAV has increased the viral genome titre when compared to a preparation comprising recombinant AAV produced by a corresponding method not comprising a step of performing depth filtration; and/or
(iii) the step of depth filtration increases the viral genome titre of the preparation comprising recombinant AAV by at least 5%, at least 10%, at least 15%, at least 20%, or at least 25%, when compared to a preparation comprising recombinant AAV produced by a corresponding method or use not comprising the step of depth filtration.

30. The method of claim 29, wherein the viral genome titre is measured by qPCR or ddPCR and/or wherein the capsid titre is measured by ELISA.

31. The method of claim 19, further comprising:
(i) purifying the recombinant AAV; and/or
(ii) formulating the preparation comprising recombinant AAV with a pharmaceutically acceptable excipient.

32. A method for increasing the viral genome titre and/or capsid titre of a preparation comprising recombinant adeno-associated virus (AAV), wherein the method comprises a step of performing mechanical lysis on mammalian producer cells comprising the recombinant AAV at a pressure between 10 and 30, preferably between 15 and 30 kilo pounds per square inch (kpsi).

33. The method of claim 32, wherein the preparation comprises recombinant AAV by at least 10%, at least 20%, at least 30%, at least 40%, at least 50%, or at least 60% increased viral genome titre and/or increased capsid titre by performing the step of mechanical lysis, when compared to a preparation comprising recombinant AAV produced by a corresponding method or use comprising a step of performing mechanical lysis at a pressure of 5 kpsi.

34. The method of claim 32, wherein performing the step of mechanical lysis comprises carrying out microfluidisation.

35. The method of claim 33, wherein the microfluidisation consists of one pass.


SEQUENCE LISTING (3 sequences)

SEQ ID NO: 1 - DNA, 4679 bp, Adeno associated virus type 2
  source 1..4679: mol_type = genomic DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 2 - DNA, 35938 bp, Adenovirus type 5
  source 1..35938: mol_type = genomic DNA
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaaacgaatagatgccatgaaccacccaactttccccgcgcccgctatgcttccactgcaacaagttgttgccggcggctttgtcccagccaatcagcctcgccccacttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 3 - DNA, 35937 bp, Adenovirus type 2
  source 1..35937: mol_type = genomic DNA
catcatcataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgccggatgtggtaaaagtgacgtttttggtgtgcgccggtgtatacgggaagtgacaattttcgcgcggttttaggcggatgttgtagtaaatttgggcgtaaccaagtaatgtttggccattttcgcgggaaaactgaataagaggaagtgaaatctgaataattctgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgcgcagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgagtctgtaatgttggcggtgcaggaagggattgacttattcacttttccgccggcgcccggttctccggagccgcctcacctttcccggcagcccgagcagccggagcagagagccttgggtccggtttctatgccaaaccttgtgccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtcggtgatagagtggtgggtttggtgtggtaatttttttttaatttttacagttttgtggtttaaagaattttgtattgtgattttttaaaaggtcctgtgtctgaacctgagcctgagcccgagccagaaccggagcctgcaagacctacccggcgtcctaaattggtgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagtctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcctcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggtggtgagacacaagaatcgcctgctactgttgtcttccgtccgcccggcaataataccgacggaggagcaacagcaggaggaagccaggcggcggcggcggcaggagcagagcccatggaacccgagagccggcctggaccctcgggaatgaatgttgtacaggtggctgaactgtttccagaactgagacgcattttaaccattaacgaggatgggcaggggctaaagggggtaaagagggagcggggggcttctgaggctacagaggaggctaggaatctaacttttagcttaatgaccagacaccgtcctgagtgtgttacttttcagcagattaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagattagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtgaggtttactggtcccaattttagcggtacggttttcctggccaataccaatcttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctgtttgaaaggtgtaccttgggtatcctgtctgagggtaactccagggtgcgccacaatgtggcctccgactgtggttgcttcatgctagtgaaaagcgtggctgtgattaagcataacatggtgtgtggcaactgcgaggacagggcctctcagatgctgacctgctcggacggcaactgtcacttgctgaagaccattcacgtagccagccactctcgcaaggcctggccagtgtttgagcacaacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtctcatgtagttttgtatctgttttgcagcagccgccgccatgagcgccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaccagactctgtttggattttgatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccgagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaaccgtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgacttgcatgttttccctgaccaaatgcgccagaaggcgctcgccgcccagcgatagcagttcttgcaaggaagcaaagtttttcaacggtttgaggccgtccgccgtaggcatgcttttgagcgtttgaccaagcagttccaggcggtcccacagctcggtcacgtgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttaagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacgaaggctcgcgtccaggccagcacgaaggaggctaagtgggaggggtagcggtcgttgtccactagggggtccactcgctccagggtgtgaagacacatgtcgccctcttcggcatcaaggaaggtgattggtttataggtgtaggccacgtgaccgggtgttcctgaaggggggctataaaagggggtgggggcgcgttcgtcctcactctcttccgcatcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgaacagaatggcggtagtgggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctaaccatgactttgaggtactggtatttgaagtcagtgtcgtcgcatccgccctgctcccagagcaaaaagtccgtgcgctttttggaacgcgggtttggcagggcgaaggtgacatcgttgaaaagtatctttcccgcgcgaggcataaagttgcgtgtgatgcggaagggtcccggcacctcggaacggttgttaattacctgggcggcgagcacgatctcgtcgaagccgttgatgttgtggcccacgatgtaaagttccaagaagcgcggggtgcccttgatggagggcaattttttaagttcctcgtaggtgagctcctcaggggagctgagcccgtgttctgacagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggtccacggctaggtctcgcgcggcggtcaccagaggctcatctccgccgaacttcataaccagcatgaagggcacgagctgcttcccaaaggcccccatccaagtataggtctctacatcgtaggtgacaaagagacgctcggtgcgaggatgcgagccgatcgggaagaactggatctcccgccaccagttggaggagtggctgttgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttatggtggatcggaccaccacgccgcgcgagcccaaagtccagatgtccgcgcgcggcggtcggagcttgatgacaacatcgcgcagatgggagctgtccatggtctggagctcccgcggcgacaggtcaggcgggagctcctgcaggtttacctcgcatagccgggtcagggcgcgggctaggtccaggtgatacctgatttccaggggctggttggtggcggcgtcgatgacttgcaagaggccgcatccccgcggcgcgactacggtaccgcgcggcgggcggtgggccgcgggggtgtccttggatgatgcatctaaaagcggtgacgcgggcgggcccccggaggtagggggggctcgggacccgccgggagagggggcaggggcacgtcggcgccgcgcgcgggcaggagctggtgctgcgcgcggaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatttcggccatgaactgctcgatctcttcctcctggagatctccgcgtccggctcgctccacggtggcggcgaggtcgttggagatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcaatctcctcttccataagggcctccccttcttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccgtgcggcagggatacggcgctaacgatgcatctcaacaattgttgtgtaggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctacggcggcggcggagtttggccgtaggtggcgccctcttcctcccatgcgtgtgaccccgaagcccctcatcggctgaagcagggccaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagcccttgagtcaaagacgtagtcgttgcaagtccgcaccaggtactgatatcccaccaaaaagtgcggcggcggctggcggtagaggggccagcgtagggtggccggggctccgggggcgaggtcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtgaggcgtgcgcagtcgttgacgctctagaccgtgcaaaaggagagcctgtaagcgggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttggcttccttccaggcgcggcggctgctgcgctagcttttttggccactggccgcgcgcggcgtaagcggttaggctggaaagcgaaagcattaagtggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctccccttctcctaccgcgtcaggaggggcaacatccgcggctgacgcggcggcagatggtgattacgaacccccgcggcgccgggcccggcactacctggacttggaggagggcgagggcctggcgcggctaggagcgccctctcctgagcgacacccaagggtgcagctgaagcgtgacacgcgcgaggcgtacgtgccgcggcagaacctgtttcgcgaccgcgagggagaggagcccgaggagatgcgggatcgaaagttccacgcagggcgcgagttgcggcatggcctgaaccgcgagcggttgctgcgcgaggaggactttgagcccgacgcgcggaccgggattagtcccgcgcgcgcacacgtggcggccgccgacctggtaaccgcgtacgagcagacggtgaaccaggagattaactttcaaaaaagctttaacaaccacgtgcgcacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacattctgcagagcatagtggtgcaggagcgcagcttgagcctggctgacaaggtggccgccattaactattccatgctcagtctgggcaagttttacgcccgcaagatataccataccccttacgttcccatagacaaggaggtaaagatcgaggggttctacatgcgcatggcgttgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgtaaccctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgatgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtccgggccagactattttttccagaccagtagacaaggcctgcagaccgtaaacctgagccaggctttcaagaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctgaactacctgctgaccaaccggcggcagaagatcccctcgttgcacagtttaaacagcgaggaggagcgcatcttgcgctatgtgcagcagagcgtgagccttaacctgatgcgcgacggggtaacgcccagcgtggcgctggacatgaccgcgcgcaacatggaaccgggcatgtatgcctcaaaccggccgtttatcaatcgcctaatggactacttgcatcgcgcggccgccgtgaaccccgagtatttcaccaatgccatcttgaacccgcactggctaccgccccctggtttctacaccgggggatttgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgcgagtagcccatttccaagcttgatagggtcttttaccagcactcgcaccacccgcccgcgcctgctgggcgaggaggagtacctaaacaactcgctgctgcagccgcagcgcgaaaagaacctgcctccggcatttcccaacaacgggatagagagcctagtggacaagatgagtagatggaagacgtatgcgcaggagcacagggatgtgcccggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaaaaaagcatgatgcaaaataaaaaactcaccaaggccatggcaccgagcgttggttttcttgtattccccttagtatgcagcgcgcggcgatgtatgaggaaggtcctcctccctcctacgagagcgtggtgagcgcggcgccagtggcggcggcgctgggttcccccttcgatgctcccctggacccgccgtttgtgcctccgcggtacctgcggcctaccggggggagaaacagcatccgttactctgagttggcacccctattcgacaccacccgtgtgtaccttgtggacaacaagtcaacggatgtggcatccctgaactaccagaacgaccacagcaactttctaaccacggtcattcaaaacaatgactacagcccgggggaggcaagcacacagaccatcaatcttgacgaccgttcgcactggggcggcgacctgaaaaccatcctgcataccaacatgccaaatgtgaacgagttcatgtttaccaataagtttaaggcgcgggtgatggtgtcgcgctcgcttactaaggacaaacaggtggagctgaaatatgagtgggtggagttcacgctgcccgagggcaactactccgagaccatgaccatagaccttatgaacaacgcgatcgtggagcactacttgaaagtgggcaggcagaacggggttctggaaagcgacatcggggtaaagtttgacacccgcaacttcagactggggtttgacccagtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgacctggagggtggtaacattcccgcactgttggatgtggacgcctaccaggcaagcttaaaagatgacaccgaacagggcggggatggcgcaggcggcggcaacaacagtggcagcggcgcggaagagaactccaacgcggcagccgcggcaatgcagccggtggaggacatgaacgatcatgccattcgcggcgacacctttgccacacgggcggaggagaagcgcgctgaggccgaggcagcggcagaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccgggatccgctcatggaccctcctttgcactcctgacgtaacctgcggctcggagcaggtctactggtcgttgccagacatgatgcaagaccccgtgaccttccgctccacgagccagatcagcaactttccggtggtgggcgccgagctgttgcccgtgcactccaagagcttctacaacgaccaggccgtctactcccagctcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaaacatgtccatccttatatcgcccagcaataacacaggctggggcctgcgcttcccaagcaagatgtttggcggggcaaagaagcgctccgaccaacacccagtgcgcgtgcgcgggcactaccgcgcgccctggggcgcgcacaaacgcggccgcactgggcgcaccaccgtcgatgacgccattgacgcggtggtggaggaggcgcgcaactacacgcccacgccgccaccagtgtccacagtggacgcggccattcagaccgtggtgcgcggagcccggcgttatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtactgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgatgaacttgacgacgaggtggaactgctgcacgcaaccgcgcccaggcggcgggtacagtggaaaggtcgacgcgtaagacgtgttttgcgacccggcaccaccgtagtttttacgcccggtgagcgctccacccgcacctacaagcgcgtgtatgatgaggtgtacggcgacgaggacctgcttgagcaggccaacgagcgcctcggggagtttgcctacggaaagcggcataaggacatgttggcgttgccgctggacgagggcaacccaacacctagcctaaagcccgtgacactgcagcaggtgctgcccacgcttgcaccgtccgaagaaaagcgcggcctaaagcgcgagtctggtgacttggcacccaccgtgcagctgatggtacccaagcgccagcgactggaagatgtcttggaaaaaatgaccgtggagcctgggctggagcccgaggtccgcgtgcggccaatcaagcaggtggcaccgggactgggcgtgcagaccgtggacgttcagatacccaccaccagtagcactagtattgccactgccacagagggcatggagacacaaacgtccccggttgcctcggcggtggcagatgccgcggtgcaggcggccgctgcggccgcgtccaaaacctctacggaggtgcaaacggacccgtggatgtttcgcgtttcagccccccggcgcccgcgccgttccaggaagtacggcaccgccagcgcactactgcccgaatatgccctacatccttccatcgcgcctacccccggctatcgtggctacacctaccgccccagaagacgagcgactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggagtctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccattaagaactatggcagcaaggcctggaacagcagcacaggccagatgctgagggacaagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcggcccgacagggaagaaactctggtgacgcaaatagatgagcctccctcgtacgaggaggcactaaagcaaggcctgcccaccacccgtcccatcgcgcccatggctaccggagtgctgggccagcacacacctgtaacgctggacctgcctccccccgctgacacccagcagaaacctgtgctgccagggccgtccgccgttgttgtaacccgccctagccgcgcgtccctgcgccgtgccgccagcggtccgcgatcgatgcggcccgtagccagtggcaactggcaaagcacactgaacagcatcgtgggtctgggggtgcaatccctgaagcgccgacgatgcttctaaatagctaacgtgtcgtatgtgtcatgtatgcgtccatgtcgccgccagaggagctgctgagccgccgtgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcacctacgcacgacgtaaccacagaccggtcccagcgtttgacgctgcggttcatccctgtggaccgcgaggataccgcgtactcgtacaaagcgcggttcaccctggctgtgggtgacaaccgtgtgcttgatatggcttccacgtactttgacatccgcggcgtgctggacagggggcctacttttaagccctactccggcactgcctacaacgctctagctcccaagggcgctcctaactcctgtgagtgggaacaaaccgaagatagcggccgggcagttgccgaggatgaagaagaggaagatgaagatgaagaagaggaagaagaagagcaaaacgctcgagatcaggctactaagaaaacacatgtctatgcccaggctcctttgtctggagaaacaattacaaaaagcgggctacaaataggatcagacaatgcagaaacacaagctaaacctgtatacgcagatccttcctatcaaccagaacctcaaattggcgaatctcagtggaacgaagctgatgctaatgcggcaggagggagagtgcttaaaaaaacaactcccatgaaaccatgctatggatcttatgccaggcctacaaatccttttggtggtcaatccgttctggttccggatgaaaaaggggtgcctcttccaaaggttgacttgcaattcttctcaaatactacctctttgaacgaccggcaaggcaatgctactaaaccaaaagtggttttgtacagtgaagatgtaaatatggaaaccccagacacacatctgtcttacaaacctggaaaaggtgatgaaaattctaaagctatgttgggtcaacaatctatgccaaacagacccaattacattgctttcagggacaattttattggcctaatgtattataacagcactggcaacatgggtgttcttgctggtcaggcatcgcagctaaatgccgtggtagatttgcaagacagaaacacagagctgtcctatcaactcttgcttgattccataggtgatagaaccagatatttttctatgtggaatcaggctgtagacagctatgatccagatgttagaatcattgaaaaccatggaactgaggatgaattgccaaattattgttttcctcttgggggtattggggtaactgacacctatcaagctattaaggctaatggcaatggctcaggcgataatggagatactacatggacaaaagatgaaacttttgcaacacgtaatgaaataggagtgggtaacaactttgccatggaaattaacctaaatgccaacctatggagaaatttcctttactccaatattgcgctgtacctgccagacaagctaaaatacaaccccaccaatgtggaaatatctgacaaccccaacacctacgactacatgaacaagcgagtggtggctcccgggcttgtagactgctacattaaccttggggcgcgctggtctctggactacatggacaacgttaatccctttaaccaccaccgcaatgcgggcctccgttatcgctccatgttgttgggaaacggccgctacgtgccctttcacattcaggtgccccaaaagttttttgccattaaaaacctcctcctcctgccaggctcatatacatatgaatggaacttcaggaaggatgttaacatggttctgcagagctctctgggaaacgatcttagagttgacggggctagcattaagtttgacagcatttgtctttacgccaccttcttccccatggcccacaacacggcctccacgctggaagccatgctcagaaatgacaccaacgaccagtcctttaatgactacctttccgccgccaacatgctataccccatacccgccaacgccaccaacgtgcccatctccatcccatcgcgcaactgggcagcatttcgcggttgggccttcacacgcttgaagacaaaggaaaccccttccctgggatcaggctacgacccttactacacctactctggctccataccataccttgacggaaccttctatcttaatcacacctttaagaaggtggccattacctttgactcttctgttagctggccgggcaacgaccgcctgcttactcccaatgagtttgagattaaacgctcagttgacggggagggctacaacgtagctcagtgcaacatgaccaaggactggttcctggtgcagatgttggccaactacaatattggctaccagggcttctacattccagaaagctacaaggaccgcatgtactcgttcttcagaaacttccagcccatgagccggcaagtggttgacgatactaaatacaaggagtatcagcaggttggaattcttcaccagcataacaactcaggattcgtaggctacctcgctcccaccatgcgcgagggacaggcttaccccgccaacgtgccctacccactaataggcaaaaccgcggttgacagtattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgagaccgtgtacctgcgcacgcccttctcggccggcaacgccacaacataaaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcgctcaaaaacatgctacctctttgagccctttggcttttctgaccaacgactcaagcaggtttaccagtttgagtacgagtcactcctgcgccgtagcgccattgcttcttcccccgaccgctgtataacgctggaaaagtccacccaaagcgtgcaggggcccaactcggccgcctgtggactattctgctgcatgtttctccacgcctttgccaactggccccaaactcccatggatcacaaccccaccatgaaccttattaccggggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtggcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgaaaatcagagggcgaggtattcagctcaacgacgagtcggtgagctcctctcttggtctccgtccggacgggacatttcagatcggcggcgctggccgctcttcatttacgccccgtcaggcgatcctaactctgcagacctcgtcctcggagccgcgctccggaggcattggaactctacaatttattgaggagttcgtgccttcggtttacttcaaccccttttctggacctcccggccactacccggaccagtttattcccaactttgacgcggtgaaagactcggcggacggctacgactgaatgaccagtggagaggcagagcgactgcgcctgacacacctcgaccactgccgccgccacaagtgctttgcccgcggctccggtgagttttgttactttgaattgcccgaagagcatatcgagggcccggcgcacggcgtccggctcaccacccaggtagagcttacacgtagcctgattcgggagtttaccaagcgccccctgctagtggagcgggagcggggtccctgtgttctgaccgtggtttgcaactgtcctaaccctggattacatcaagatctttgttgtcatctctgtgctgagtataataaatacagaaattagaatctactggggctcctgtcgccatcctgtgaacgccaccgtttttacccacccaaagcagaccaaagcaaacctcacctccggtttgcacaagcgggccaataagtaccttacctggtactttaacggctcttcatttgtaatttacaacagtttccagcgagacgaagtaagtttgccacacaaccttctcggcttcaactacaccgtcaagaaaaacaccaccaccaccaccctcctcacctgccgggaacgtacgagtgcgtcaccggttgctgcgcccacacctacagcctgagcgtaaccagacattactcccatttttccaaaacaggaggtgagctcaactcccggaactcaggtcaaaaaagcattttgcggggtgctgggattttttaattaagtatatgagcaattcaagtaactctacaagcttgtctaatttttctggaattggggtcggggttatccttactcttgtaattctgtttattcttatactagcacttctgtgccttagggttgccgcctgctgcacgcacgtttgtacctattgtcagctttttaaacgctgggggcaacatccaagatgaggtacatgattttaggcttgctcgcccttgcggcagtctgcagcgctgccaaaaaggttgagtttaaggaaccagcttgcaatgttacatttaaatcagaagctaatgaatgcactactcttataaaatgcaccacagaacatgaaaagcttattattcgccacaaagacaaaattggcaagtatgctgtatatgctatttggcagccaggtgacactaacgactataatgtcacagtcttccaaggtgaaaatcgtaaaacttttatgtataaatttccattttatgaaatgtgcgatattaccatgtacatgagcaaacagtacaagttgtggcccccacaaaagtgtttagagaacactggcaccttttgttccaccgctctgcttattacagcgcttgctttggtatgtaccttactttatctcaaatacaaaagcagacgcagttttattgatgaaaagaaaatgccttgattttccgcttgcttgtattcccctggacaatttactctatgtgggatatgctccaggcgggcaagattatacccacaaccttcaaatcaaactttcctggacgttagcgcctgatttctgccagcgcctgcactgcaaatttgatcaaacccagcttcagcttgcctgctccagagatgaccggctcaaccatcgcgcccacaacggactatcgcaacaccactgctaccggactaacatctgccctaaatttaccccaagttcatgcctttgtcaatgactgggcgagcttggacatgtggtggttttccatagcgcttatgtttgtttgccttattattatgtggcttatttgttgcctaaagcgcagacgcgccagaccccccatctataggcctatcattgtgctcaacccacacaatgaaaaaattcatagattggacggtctgaaaccatgttctcttcttttacagtatgattaaatgagacatgattcctcgagttcttatattattgacccttgttgcgcttttctgtgcgtgctctacattggccgcggtcgctcacatcgaagtagattgcatcccacctttcacagtttacctgctttacggatttgtcacccttatcctcatctgcagcctcgtcactgtagtcatcgccttcattcagttcattgactgggtttgtgtgcgcattgcgtacctcaggcaccatccgcaatacagagacaggactatagctgatcttctcagaattctttaattatgaaacggagtgtcatttttgttttgctgattttttgcgccctacctgtgctttgctcccaaacctcagcgcctcccaaaagacatatttcctgcagattcactcaaatatggaacattcccagctgctacaacaaacagagcgatttgtcagaagcctggttatacgccatcatctctgtcatggttttttgcagtaccatttttgccctagccatatatccataccttgacattggctggaatgccatagatgccatgaaccaccctactttcccagtgcccgctgtcataccactgcaacaggttattgccccaatcaatcagcctcgccccccttctcccacccccactgagattagctactttaatttgacaggtggagatgactgaatctctagatctagaattggatggaattaacaccgaacagcgcctactagaaaggcgcaaggcggcgtccgagcgagaacgcctaaaacaagaagttgaagacatggttaacctacaccagtgtaaaagaggtatcttttgtgtggtcaagcaggccaaacttacctacgaaaaaaccactaccggcaaccgcctcagctacaagctacccacccagcgccaaaaactggtgcttatggtgggagaaaaacctatcaccgtcacccagcactcggcagaaacagagggctgcctgcacttcccctatcagggtccagaggacctctgcactcttattaaaaccatgtgtggtattagagatcttattccattcaactaacataaacacacaataaattacttacttaaaatcagtcagcaaatctttgtccagcttattcagcatcacctcctttccttcctcccaactctggtatctcagccgccttttagctgcaaactttctccaaagtttaaatgggatgtcaaattcctcatgttcttgtccctccgcacccactatcttcatattgttgcagatgaaacgcgccagaccgtctgaagacaccttcaaccccgtgtatccatatgacacagaaaccgggcctccaactgtgccctttcttacccctccatttgtttcacccaatggtttccaagaaagtccccctggagttctctctctacgcgtctccgaacctttggacacctcccacggcatgcttgcgcttaaaatgggcagcggtcttaccctagacaaggccggaaacctcacctcccaaaatgtaaccactgttactcagccacttaaaaaaacaaagtcaaacataagtttggacacctccgcaccacttacaattacctcaggcgccctaacagtggcaaccaccgctcctctgatagttactagcggcgctcttagcgtacagtcacaagccccactgaccgtgcaagactccaaactaagcattgctactaaagggcccattacagtgtcagatggaaagctagccctgcaaacatcagcccccctctctggcagtgacagcgacacccttactgtaactgcatcacccccgctaactactgccacgggtagcttgggcattaacatggaagatcctatttatgtaaataatggaaaaataggaattaaaataagcggtcctttgcaagtagcacaaaactccgatacactaacagtagttactggaccaggtgtcaccgttgaacaaaactcccttagaaccaaagttgcaggagctattggttatgattcatcaaacaacatggaaattaaaacgggcggtggcatgcgtataaataacaacttgttaattctagatgtggattacccatttgatgctcaaacaaaactacgtcttaaactggggcagggacccctgtatattaatgcatctcataacttggacataaactataacagaggcctatacctttttaatgcatcaaacaatactaaaaaactggaagttagcataaaaaaatccagtggactaaactttgataatactgccatagctataaatgcaggaaagggtctggagtttgatacaaacacatctgagtctccagatatcaacccaataaaaactaaaattggctctggcattgattacaatgaaaacggtgccatgattactaaacttggagcgggtttaagctttgacaactcaggggccattacaataggaaacaaaaatgatgacaaacttaccctgtggacaaccccagacccatctcctaactgcagaattcattcagataatgactgcaaatttactttggttcttacaaaatgtgggagtcaagtactagctactgtagctgctttggctgtatctggagatctttcatccatgacaggcaccgttgcaagtgttagtatattccttagatttgaccaaaacggtgttctaatggagaactcctcacttaaaaaacattactggaactttagaaatgggaactcaactaatgcaaatccatacacaaatgcagttggatttatgcctaaccttctagcctatccaaaaacccaaagtcaaactgctaaaaataacattgtcagtcaagtttacttgcatggtgataaaactaaacctatgatacttaccattacacttaatggcactagtgaatccacagaaactagcgaggtaagcacttactctatgtcttttacatggtcctgggaaagtggaaaatacaccactgaaacttttgctaccaactcttacaccttctcctacattgcccaggaataaagaatcgtgaacctgttgcatgttatgtttcaacgtgtttatttttcaattgcagaaaatttcaagtcatttttcattcagtagtatagccccaccaccacatagcttatattgatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcgcttaagttcatgtcgctgtccagctgctgagccacaggctgctgtccaacttgcggttgctcaacgggcggcgaaggggaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgtcgcttagctcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgttttttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaactgccctcacgtccaagtggacgtaaaggctaaacccttcagggtgaatctcctctataaacattccagcaccttcaaccatgcccaaataattttcatctcgccaccttatcaatatgtctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagcccctatgtaagcttgttgcatgggcggcgatataaaatgcaaggtgctgctcaaaaaatcaggcaaagcctcgcgcaaaaaagcaagcacatcgtagtcatgctcatgcagataaaggcaggtaagttccggaaccaccacagaaaaagacaccatttttctctcaaacatgtctgcgggttcctgcattaaacacaaaataaaataacaaaaaaaaacatttaaacattagaagcctgtcttacaacaggaaaaacaacccttataagcataagacggactacggccatgccggcgtgaccgtaaaaaaactggtcaccgtgattaaaaagcaccaccgacagttcctcggtcatgtccggagtcataatgtaagactcggtaaacacatcaggttggttaacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaaacctattaaaaaacaccactcgacacggcaccagctcaatcagtcacagtgtaaaaagggccaagtacagagcgagtatatataggactaaaaaatgacgtaacggttaaagtccacaaaaaacacccagaaaaccgcacgcgaacctacgcccagaaacgaaagccaaaaaacccacaacttcctcaaatcttcacttccgttttcccacgatacgtcacttcccattttaaaaaaactacaattcccaatacatgcaagttactccgccctaaaacctacgtcacccgccccgttcccacgccccgcgccacgtcacaaactccaccccctcattatcatattggcttcaatccaaaataaggtatattatgatgatg